(19) United States
(12) Patent Application Publication
(10) Pub. No.: US 2025/0073067 A1
(43) Pub. Date: Mar. 6, 2025

(54) SYSTEM AND METHOD FOR A SURGICAL HANDPIECE AND MULTI-MODAL TIP

(71) Applicant: Alcon Inc., Fribourg (CH)

(72) Inventors: Kurt D. LEUKANECH, Laguna Nigel, CA (US); Satish YALAMANCHILI, Irvine, CA (US); Mikhail OVCHINNIKOV, Dana Point, CA (US)

(21) Appl. No.: 18/810,796

(22) Filed: Aug. 21, 2024

Related U.S. Application Data
(60) Provisional application No. 63/578,802, filed on Aug. 25, 2023.

(51) Int. Cl. A61F 9/007; A61M 1/00

(52) U.S. Cl. CPC A61F 9/00745; A61M 1/774; A61M 2205/3334

(57) ABSTRACT

In certain embodiments, a surgical handpiece system includes a phacoemulsification tip, a fluid management system, an ultrasonic drive system, and a controller. The phacoemulsification tip is attached to a shaft. The fluid management system is configured to manage inflow and outflow of fluid from an ocular surgical site. The ultrasonic drive system is configured to generate torsional and longitudinal vibrations in the phacoemulsification tip. The controller is configured to analyze applied voltage and current to the ultrasonic drive system using Fast Fourier Transforms (FFTs), and tune applied frequencies of the torsional and longitudinal vibrations according to at least one resonant frequency.

### CROSS-REFERENCE TO RELATED APPLICATIONS

This application claims the benefit of U.S. Provisional Application Ser. No. 63/578,802 (filed on Aug. 25, 2023), the content of which is incorporated herein by reference in its entirety.

### BACKGROUND

A typical ultrasonic surgical device/system suitable for ophthalmic procedures includes an ultrasonically driven handpiece, an attached hollow working tip or cutting needle, an irrigating sleeve and an electronic control console. The handpiece assembly is attached to the control console by an electric cable and flexible tubing. Through the electric cable, the console varies the power level transmitted by the handpiece to the attached working tip, and the flexible tubing is used to supply irrigation fluid to and draw aspiration fluid from the eye through the handpiece assembly.

In some examples of such devices, the operative part of the handpiece is a centrally-located, hollow resonating bar or horn directly attached to a set of piezoelectric crystals that form a piezoelectric element assembly. The crystals supply the required ultrasonic vibration needed to drive both the horn and the attached working tip during phacoemulsification and are controlled by the console. The crystal/horn assembly is suspended within the hollow body or shell of the handpiece. The handpiece body terminates in a reduced diameter portion or nosecone at the body's distal end. The nosecone is externally threaded to accept the irrigation sleeve. Likewise, the horn bore is internally threaded at its distal end to receive the external threads of the working tip. The irrigation sleeve also has an internally threaded bore that is screwed onto the external threads of the nosecone. The working tip is adjusted so that the tip projects only a predetermined amount past the open end of the irrigating sleeve.

When used to perform phacoemulsification, the ends of the working tip and irrigating sleeve are inserted into a small incision of predetermined width in the cornea, sclera, or other location in the eye tissue in order to gain access to the anterior chamber of the eye. The working tip is ultrasonically vibrated along its longitudinal axis within the irrigating sleeve by the crystal-driven ultrasonic horn, thereby emulsifying upon contact the selected tissue in situ. The hollow bore of the working tip communicates with the bore in the horn which in turn communicates with the aspiration line from the handpiece to the console. A reduced pressure or vacuum source in the console draws or aspirates the emulsified tissue from the eye through the open end of the working tip, the bore of the working tip, the horn bore, and the aspiration line and into a collection device. The aspiration of emulsified tissue is aided by a saline flushing solution or irrigant that is injected into the surgical site through the small annular gap between the inside surface of the irrigating sleeve and the outside surface of the working tip. In some cases, the hand piece assembly is attached to the control console by an electric cable and flexible tubing. Through the electric cable, the console varies the power level transmitted by the hand piece to the attached cutting needle and the flexible tubing supply irrigation fluid to and draw aspiration fluid from the eye through the hand piece assembly.

The operative part of the hand piece is a centrally located, hollow resonating bar or horn directly attached to a set of piezoelectric crystals. The crystals supply the required ultrasonic vibration needed to drive both the horn and the attached cutting needle during phacoemulsification and are controlled by the console. The crystal/horn assembly is suspended within the hollow body or shell of the hand piece by flexible mountings. The hand piece body terminates in a reduced diameter portion or nosecone at the body's distal end. The nosecone is externally threaded to accept the irrigation sleeve. Likewise, the horn bore is internally threaded at its distal end to receive the external threads of the cutting needle. The irrigation sleeve also has an internally threaded bore that is screwed onto the external threads of the nosecone. The cutting needle is adjusted so that the needle projects only a predetermined amount past the open end of the irrigating sleeve.

In use, the ends of the cutting needle and irrigating sleeve are inserted into a small incision of predetermined width in the cornea or sclera. The cutting needle is ultrasonically vibrated along its longitudinal axis within the irrigating sleeve by the crystal-driven ultrasonic horn, thereby emulsifying the selected tissue in situ. The hollow bore of the cutting needle communicates with the bore in the horn that in turn communicates with the aspiration line from the hand piece to the console. A reduced pressure or vacuum source in the console draws or aspirates the emulsified tissue from the eye through the open end of the cutting needle, the cutting needle and horn bores and the aspiration line and into a collection device. The aspiration of emulsified tissue is aided by a saline solution or irrigating solution that is injected into the surgical site through the small annular gap between the inside surface of the irrigating sleeve and the cutting needle. In some cases, the cutting needle, which is typically made of titanium or stainless steel, may damage eye structures. The distal end of the cutting needle may also sometimes be sharper than is necessary to perform cataract removal.

### SUMMARY

In certain embodiments, a surgical handpiece system includes a phacoemulsification tip, a fluid management system, an ultrasonic drive system, and a controller. The phacoemulsification tip is attached to a shaft. The fluid management system is configured to manage inflow and outflow of fluid from an ocular surgical site. The ultrasonic drive system is configured to generate torsional and longitudinal vibrations in the phacoemulsification tip. The controller is configured to analyze applied voltage and current to the ultrasonic drive system using Fast Fourier Transforms (FFTs), and tune applied frequencies of the torsional and longitudinal vibrations according to at least one resonant frequency.

In certain embodiments, a method of operating an ultrasonic handpiece includes applying control signals to drive a piezoelectric element assembly of the ultrasonic handpiece simultaneously in a first mode of oscillation and a second mode of oscillation; generating feedback of a resulting oscillation of the piezoelectric element assembly in the first mode and the second mode; and adjusting, based on the feedback, the control signals so that the resulting oscillation in the first mode and the resulting oscillation in the second mode are each approximately at a respective resonant frequency.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIGS. 1A, 1B present a perspective view and block diagram of a bi-modal ultrasonic handpiece surgical system, in accordance with some embodiments;

FIG. 2 is a prior art timing diagram for operating surgical handpieces;

FIG. 3 illustrates a timing diagram for operating an ultrasonic handpiece using the controller of FIG. 1B, in accordance with some embodiments;

FIG. 4 is a detailed block diagram of a bi-modal ultrasonic handpiece system, in accordance with some embodiments;

FIG. 5 is a detailed block diagram of the longitudinal quadrature amplitude demodulator and torsional quadrature amplitude demodulator of FIG. 4, in accordance with some embodiments;

FIG. 6 is a detailed block diagram of a bi-modal ultrasonic handpiece system, in accordance with some embodiments;

FIG. 7 is a detailed block diagram of the longitudinal quadrature phase demodulator and torsional quadrature phase demodulator of FIG. 6, in accordance with some embodiments;

FIG. 8 is a flow diagram of an example of functionality of the controller of FIG. 1B for providing multiple modes of oscillation simultaneously for an ultrasonic handpiece via a single piezoelectric element assembly in accordance with some embodiments;

FIG. 9 illustrates a balanced phacoemulsification tip with a distal end having two bends, for use with the handpiece system of FIG. 1;

FIG. 10 illustrates a surgical console connected to a handpiece through an irrigation line and an aspiration line, according to some embodiments;

FIG. 11 illustrates an ultrasonic horn attached to the balanced tip, according to an embodiment;

FIG. 12 illustrates motion of the balanced tip, according to an embodiment;

FIG. 13 illustrates a balanced tip inserted into an incision in the eye, in accordance with some embodiments;

FIG. 14 illustrates twisting vibrations and lateral vibrations relative to the balanced tip, in accordance with some embodiments;

FIG. 15 illustrates model equations for twist displacement along the z axis of the tip, in accordance with some embodiments;

FIG. 16 illustrates model equations for lateral displacement along the z axis of the tip, in accordance with some embodiments;

FIG. 17 illustrates a component (l(z)) of the modeling equations, in accordance with some embodiments;

FIGS. 18-19 illustrate embodiments of input tip shapes and corresponding output lateral displacement and twist angle along the tip length according to the model equations, in accordance with some embodiments;

FIG. 20 illustrates a flowchart of the method for determining a tip geometry, in accordance with some embodiments;

FIG. 21 illustrates a flowchart of another method for determining a tip geometry, in accordance with some embodiments;

FIG. 22 illustrates six possible balanced tips, in accordance with some embodiments;

FIG. 23 is a diagram of a hybrid tip phacoemulsification hand piece system for use with the handpiece system of FIG. 1;

FIG. 24 is a side view of a phacoemulsification needle with a polymer distal end, in accordance with some embodiments;

FIG. 25 is a perspective view of the distal end of a phacoemulsification needle, in accordance with some embodiments;

FIG. 26 is a side cross section view of the distal end of a phacoemulsification needle with an over molded distal end, in accordance with some embodiments;

FIG. 27 is a side cross section view of the distal end of a phacoemulsification needle with an over molded distal end, in accordance with some embodiments;

FIG. 28 is a side cross section view of the distal end of a phacoemulsification needle with an over molded distal end, in accordance with some embodiments; and

FIG. 29 is a side cross section view of the distal end of a phacoemulsification needle with an over molded distal end, in accordance with some embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

As used herein, the singular form of “a”, “an”, and “the” include plural references unless the context clearly dictates otherwise. As used herein, the statement that two or more parts or components are “coupled” shall mean that the parts are joined or operate together either directly or indirectly (i.e., through one or more intermediate parts or components, so long as a link occurs). As used herein, “directly coupled” means that two elements are directly in contact with each other. As used herein, “fixedly coupled” or “fixed” means that two components are coupled so as to move as one while maintaining a constant orientation relative to each other. As used herein, “operatively coupled” means that two elements are coupled in such a way that the two elements function together. It is to be understood that two elements “operatively coupled” does not require a direct connection or a permanent connection between them. As utilized herein, “substantially” means that any difference is negligible, such that any difference is within an operating tolerance that is known to persons of ordinary skill in the art and provides for the desired performance and outcomes as described in the embodiments described herein. Descriptions of numerical ranges arc endpoints inclusive.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the exemplary embodiments described herein, an embodiment showing a singular component should not be considered limiting; rather, the disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The embodiments described herein provide a multi-mode ultrasonic handpiece system for phacoemulsification. In some embodiments, the handpiece system incorporates a hollow needle combined with a tip that has at least a first and second bend. The tip is configured to enable multiple modes of motion, including, but not limited to, torsional and longitudinal, and additional modes corresponding to their applicable resonant frequencies. The handpiece's piezoelectric element assembly concurrently drives the needle and tip in more than two modes of oscillation.

A Fast Fourier Transform (FFT) based control system, in some embodiments, is responsible for generating feedback from the oscillations of the needle and tip. The system independently adjusts the frequency of each oscillation mode, allowing each to operate near its respective resonant frequency. In some embodiments, such configuration optimizes lateral displacement during high-frequency torsional vibrations, maintaining it within 5% to 25% of the displacement at the distal endpoint of the tip. Optimized displacement enhances both cutting efficiency and safety. Further, in some embodiments, the system permits variable modulation of the percentage of each mode and resonant frequency applied, providing the surgeon with precise and adaptable control. Software and/or physical modeling may be employed in some embodiments to determine optimal geometrical configurations and balance the tradeoffs between various modes of operation.

Certain embodiments described herein relate to a balanced phacoemulsification tip, which is described in more detail in U.S. Pat. No. 10,258,505, which is incorporated herein by reference in its entirety.

Certain embodiments described herein relate to a hybrid phacoemulsification tip, which is described in more detail in U.S. Pat. No. 11,185,442, which is incorporated herein by reference in its entirety.

Aspects of the present disclosure include a system to operate an ultrasonic handpiece tool that includes a piezoelectric element assembly. In some examples, the system applies control signals to drive the piezoelectric element assembly simultaneously in a first mode of oscillation and a second mode of oscillation. In some examples, the system generates feedback of a resulting oscillation of the piezoelectric element assembly in the first mode and the second mode. In some examples, based on the feedback, the system adjusts the frequency of each of the first mode and second mode, wherein the resulting oscillation of each of the first mode and the second mode is each approximately at its respective resonant frequency. In some examples, methods are disclosed for operating ultrasonic devices in a first mode of oscillation and a second mode of oscillation. In some examples, based on the feedback, the method comprises independently adjusting the frequency of each of the first mode and second mode, as needed, so that the resulting oscillation of each of the first mode and the second mode is each approximately at its respective resonant frequency.

In certain embodiments, a phacoemulsification tip may include a shaft and a cutting edge portion having at least a first and second bend. The geometry of the shaft and the at least first and second bend may be configured to result in a lateral displacement, perpendicular to the shaft during ultrasonic torsional vibration of the tip, of the shaft along its length that is less than approximately 5% to 25% (e.g., 15%) (Other thresholds may also be used) of the displacement of the distal end point of the tip. In some embodiments, the shaft may extend from the end of a conical portion (which may be, for example, approximately 12 mm (millimeters) from the distal end point of the tip) through to the first bend in the cutting edge portion (which may be, for example, approximately 5 mm from the distal end point of the tip). Other locations of the first bend are also contemplated (e.g., 3 mm, 8 mm, etc. from the distal end point of the tip). In some embodiments, a proximal end of the conical portion (i.e., the hub) may be configured to couple to an ultrasonic horn.

In certain embodiments, a phacoemulsification tip or needle has a hollow shaft with an interior surface, an exterior surface, and a distal end terminating in a distal edge, the shaft having a central bore extending there through, the central bore defined by the interior surface of the hollow shaft; and a first over mold located on the exterior surface and distal edge of the hollow shaft, the first over mold covering at least a portion of a periphery of the exterior surface of the hollow shaft, the first over mold covering the distal edge and terminating at the central bore.

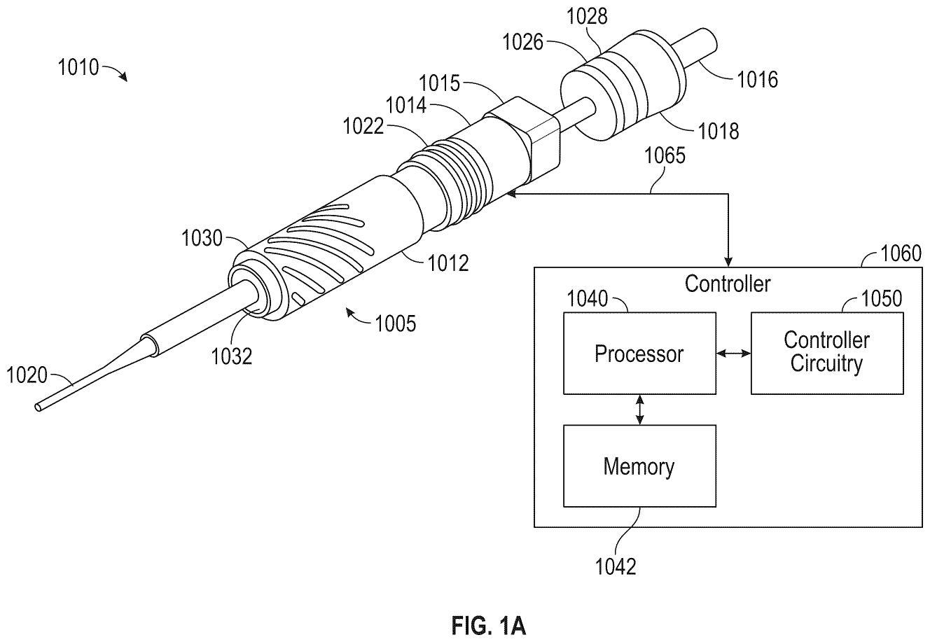

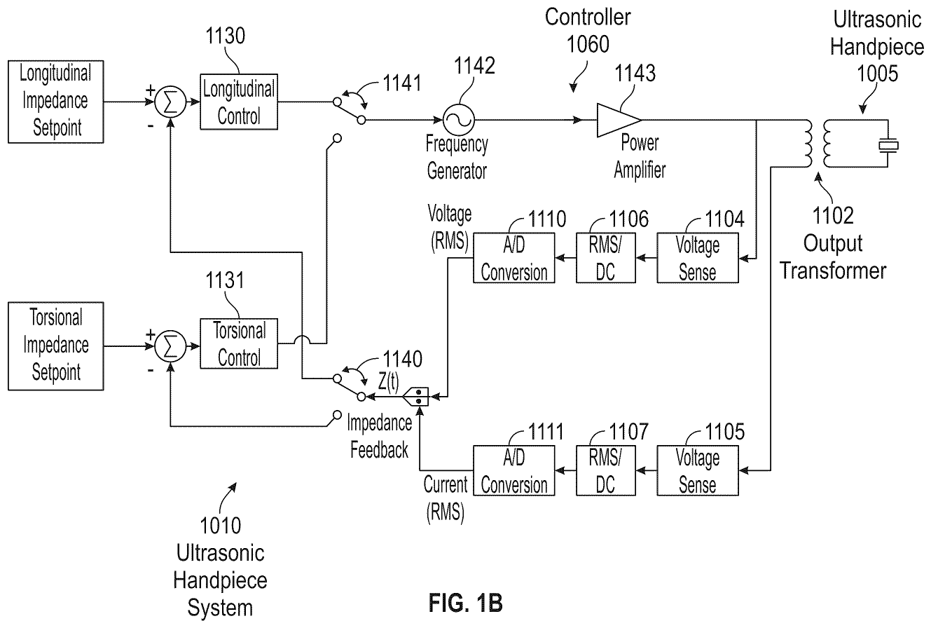

FIG. 1A is a perspective view and FIG. 1B is a block diagram of a bi-modal ultrasonic handpiece system 1010 in accordance with some embodiments. System 1010 may be used, for example, to perform phacoemulsification. FIG. 1A includes a perspective view of the handpiece 1005 with the outer case removed, and a block diagram of a controller 1060 that controls the handpiece 1005. Handpiece 1005 includes an ultrasonic horn 1012, for example made from a titanium alloy. Horn 1012 has a plurality of helical slits. A plurality (typically 1 or 2 pairs or more) of piezoelectric elements or crystals form a piezoelectric element assembly 1014. The piezoelectric elements or crystals may be ring-shaped and may be held by a compression nut 1015 against horn 1012. Some handpieces may include multiple piezoelectric element assemblies 1014 that are each physically separated from each other along the longitudinal axis of handpiece 1005, and each may form a separate assembly/packaging. Each piezoelectric element assembly 1014 may be separately electrically coupled to controller 1060.

An aspiration shaft or tube 1016 extends down the length of handpiece 1005 through horn 1012, piezoelectric element assembly 1014, nut 1015, and plug 1018 at the proximal end of handpiece 1005. Aspiration tube 1016 allows material to be aspirated through a hollow working tip 1020, which is attached to horn 1012, and through and out handpiece 1005. While hollow working tip 1020 is shown as a straight tip, other tip configurations may also be used (e.g., a bent tip). Plug 1018 seals an outer shell of handpiece 1005 fluid tight, allowing handpiece 1005 to be autoclaved without adversely affecting piezoelectric element assembly 1014. Additional grooves 1022 for sealing O-ring gaskets may be provided on horn 1012.

The location of longitudinal and torsional nodal points (the points with zero velocity of the respective mode) of handpiece 1005 are indicated on FIG. 1. The torsional node 1026 preferably is located at the proximal longitudinal node 1028, so that the torsional node 1026 and the longitudinal node 1028 are coincident, e.g., both of which are located on plug 1018. Handpiece 1005 also includes a distal longitudinal node 1030 located at reduced diameter portion 1032 of horn 1012.

Controller 1060 is generally located remote from handpiece 1005 and can be part of an electronic control console. Controller 1060 is coupled to handpiece 1005 at piezoelectric element assembly 1014 via an electric cable or connector 1065, or may be coupled via other communication means, including wirelessly. The electronic control console is further coupled to handpiece 1005 via flexible tubing in order to provide irrigation and aspiration.

Controller 1060 includes a processor 1040, a memory 1042, and a controller circuitry 1050. Processor 1040 may be any type of general purpose processor or could be a processor specifically designed for handpiece 1005, such as an application-specific integrated circuit (“ASIC”). Processor 1040 may be the same processor that operates the entire system 1010 or may be a separate processor.

Memory 1042 can be any type of storage device or non-transitory computer-readable medium, such as random access memory (“RAM”) or read-only memory (“ROM”). Memory 1042 stores instructions executed by processor 1040, including instructions to provide multiple modes (e.g., bi-modal) of oscillation simultaneously (i.e., at the same time) via a single piezoelectric element assembly 1014, and other functionality disclosed herein. Controller circuitry 1050 also provides functionality, in addition to the functionality of processor 1040, for providing multiple modes of oscillation simultaneously via a single piezoelectric element assembly 1014. In example embodiments, functionality disclosed herein can be provided by processor 1040 and memory 1042 (i.e., software based) or by controller circuitry 1050 (i.e., hardware based) or a combination of both.

The control of ultrasonic motion for a handpiece such as handpiece 1005 can be implemented by a number of different methods. One method involves a control loop which servos the frequency of the drive voltage by using the electrical impedance of the piezoelectric drive transducers as feedback. In such a method, the impedance feedback of the piezo-electric transducers is computed as the ratio of the root mean square (“RMS”) value of the transducer drive voltage to the RMS value of drive current.

In certain instances, there are advantages in utilizing two or more different modes of oscillation, such as orthogonal longitudinal modes and torsional modes, in an ultrasonic handpiece. However, each mode of oscillation has a distinct resonance of operation which must be independently controlled to maintain the optimal operational frequency in response to various influences such as loading and temperature.

In some known ultrasonic handpiece systems, the handpieces are controlled to provide an ultrasonic longitudinal motion of the cutting tip and a rotational/torsional motion of the tip. Such known ultrasonic handpiece systems for phacoemulsification may include a drive circuit that monitors both the torsional mode and the longitudinal mode and controls these modes using two different drive frequencies. The torsional drive signal is approximately 31 kHz (kilohertz) and the longitudinal drive signal is approximately 45 kHz, but these frequencies may change depending upon the piezoelectric element assemblies 1014 used and the size and shape of horn 1012 and slits 1024. The frequencies of both the longitudinal and torsional modes are tracked and controlled so that the frequencies of these motions are generally at the respective resonant frequencies when being applied.

However, known systems for providing both a longitudinal motion and a torsional motion generally alternate these motions on a single piezoelectric element assembly 1014, or use multiple different piezoelectric element assemblies 1014 for each different motion. Known systems fail to determine the resonance frequency of both modes simultaneously and fail to simultaneously make the necessary adjustments in the frequency of operations of both modes in order to maintain optimal resonant frequency for both modes in reaction to the various factors that shift the resonant frequencies, such as temperature.

FIG. 1B is a detailed block diagram of ultrasonic handpiece system 1010 in accordance with some examples. FIG. 1B illustrates details of controller 1060 that is coupled to ultrasonic handpiece 1005. In general, in FIG. 1B, controller 1060 tracks resonance by measuring the electrical impedance (i.e., a measurement of voltage and current) reflected back to a primary of an output transformer 1102. The RMS value voltage is measured by an RMS to DC (Direct Current) converter, RMS/DC 1106, and voltage sense 1104, and the current (at the same time as the voltage) is measured by RMS/DC 1107 and current sense 1105, and A/D (analog-to-digital) converters 1110 and 1111. The measurements generate an impedance feedback 1120 (“Z(t)”). Based on the measured impedance feedback, control loops 1130 and 1131 adjust the frequency to maintain resonance frequency. In some embodiments, such frequency is facilitated by frequency generator 1142, which is coupled to power amplifier 1143.

Power amplifier 1143 operates in conjunction with frequency generator 1142 by amplifying the generated frequency signals to desired power levels. Such amplified output is fed to ultrasonic handpiece 1005, driving various multi-mode vibrations in the phacoemulsification tip with optimal efficiency. In some embodiments, based on the measured impedance feedback, the control loops 1130 and 1131 may adjust the frequency to maintain resonance frequency. To achieve such fine-tuning, the frequency generator 1142 and power amplifier 1143, together generate the desired frequency signals required for multimode vibration in surgical tips, which discussed in detail further below.

The longitudinal control loop 1130 and torsional control loop 1131 provide the adjustments separately using switching functionality 1140 and 1141 where the switching is always in either a longitudinal control position as shown in FIG. 2, or the opposite positions for a torsional control position. Such switching functionality is advantageous in facilitating precise control and differentiation between the vibrational modes, enabling the handpiece to dynamically alternate between torsional and longitudinal motions as needed during the surgical procedure. Thus, controller 1060, frequency generator 1142, power amplifier 1143, and the switching functionality described above ensures the precise and seamless execution of vibrational modes (discussed below). Such advanced control system significantly enhances surgical precision, ultimately leading to improved surgical outcomes and better patient care.

With the embodiment of FIG. 1B, when operating multiple modes of oscillation, the RMS value of measured voltage and resulting current is an indication of the aggregate of all component frequencies present in the voltage and current signals being measured. As a result, with embodiments of FIG. 1B, it is not possible to independently separate the information (i.e., impedance magnitude) for each resonance to be controlled.

In some embodiments embedded within ultrasonic handpiece 1005, a temperature sensor may continuously monitor the temperature within the surgical environment and adjust frequency based on temperature in the surgical environment. The precise modulation of the frequency based on the temperature data optimizes the cutting power applied to the lens material, minimizing the energy requirements and reducing the risk of thermal injury to the delicate ocular structures. In this manner, surgeons may confidently navigate through dense cataracts or challenging surgical scenarios, wherein the handpiece's performance may be dynamically aligned with the specific needs of each patient.

For example, as the phacoemulsification process progresses, the temperature sensor embedded within ultrasonic handpiece 1005 may generate data reflecting the thermal dynamics within the eye. Such data advantageously serves as an input to, for example, controller 1060, thereby providing real-time feedback on changes in the eye's internal temperature, especially during the emulsification of dense cataractous lens material.

By leveraging such temperature feedback functionality, controller 1060 may dynamically adjust and tune the frequency applied to the phaco crystals. Such frequency modulation may be tailored to maintain the optimal cutting efficiency while mitigating any potential thermal impact on surrounding ocular tissues. This real-time adaptive approach enables controller 1060 to respond to fluctuations in temperature, ensuring that the phacoemulsification process remains both efficient and safe.

The integration of temperature feedback in adjusting the phaco crystals' frequency represents a remarkable advancement in ocular surgery. Moreover, such temperature feedback control may be applied by considering temperature data during other ocular procedures beside cataract surgery, such as fragment removal or corneal interventions. In this manner controller 1050 may continuously fine-tune the phaco crystals' frequency, adapting to the dynamic surgical environment in real-time.

FIG. 2 illustrates a prior art timing diagram for operating an ultrasonic handpiece using a controller similar to controller 1060 of FIG. 1A, but with different control functionality. In FIG. 2, torsional mode and longitudinal mode are applied separately to a single piezoelectric element. Specifically, at 201 torsional mode is applied and longitudinal mode is off (i.e., when 1140 and 1141 are switched to the torsional control). At 202, longitudinal mode is applied and torsional mode is off (i.e., when 1140 and 1141 are switched to the longitudinal control). During “dead” times 204 and 205, neither of the modes are applied and the respective mode that was applied previously is adjusted by either control loop 1130 or 1131 to maintain the respective resonant frequency based on the impedance feedback. This “dead” time is advantageous to allow the energy from one mode to decay prior to exciting the alternate mode. However, as shown in FIG. 2, because the alternate modes of oscillation are multiplexed in time as opposed to being applied simultaneously, tissue cutting efficiency is sacrificed when used, for example, to perform phacoemulsification.

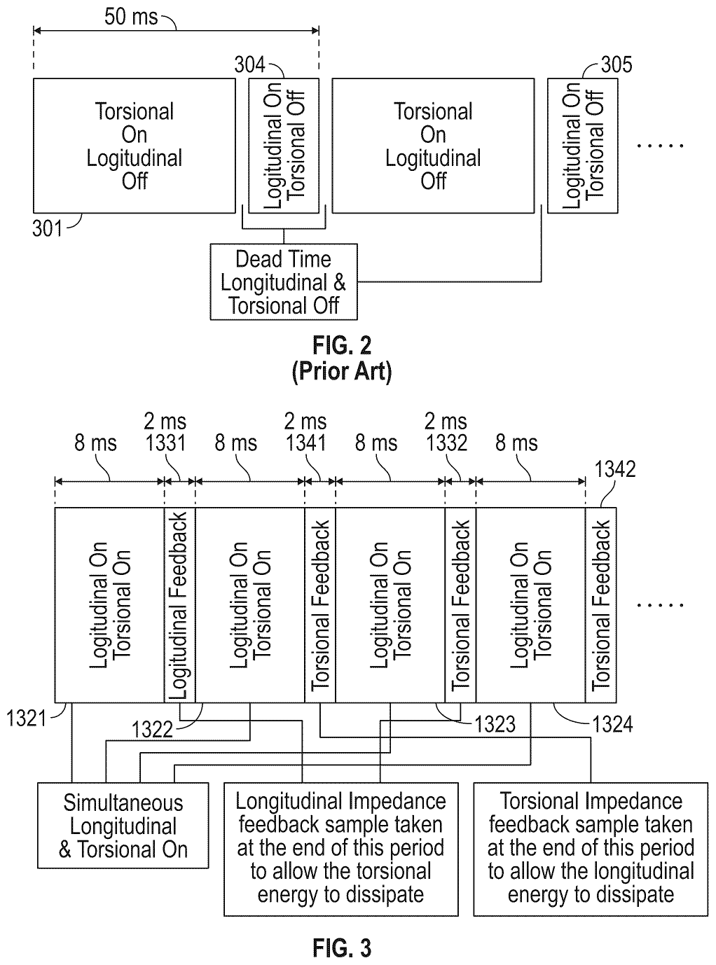

FIG. 3 illustrates a timing diagram for operating ultrasonic handpiece 1005 using controller 1060 of FIG. 2 in accordance with some embodiments. In contrast to FIG. 2, during periods 1321-1324, both longitudinal mode and torsional mode are applied simultaneously to a single piezoelectric element 1014 (i.e., exciting the piezoelectric element with both longitudinal and torsional frequencies at the same time) while maintaining independent control of each mode of oscillation. During feedback measurement periods 1331 and 1332, a longitudinal impedance feedback sample is taken while the longitudinal mode is turned on and the torsional mode is off to allow the torsional energy to dissipate. Similarly, during feedback measurement periods 1341 and 1342, a torsional impedance feedback sample is taken while the torsional mode is turned on and the longitudinal mode is off to allow the longitudinal energy to dissipate.

FIG. 3 provides for the timing of the feedback such that the simultaneous application of multiple modes of oscillation can be maintained with a high duty cycle. In the timing shown in FIG. 3, the simultaneous application of multiple modes of oscillation on a single piezoelectric element 1014 is active approximately 80% of the time. In this operational mode there is an alternation in time between longer periods of the simultaneous drive periods 1321-1324 followed by brief periods of feedback measurement periods 1331, 1332, 1341, and 1342. Each feedback period is used to sample the feedback of one mode of oscillation, and alternating feedback periods alternate between the modes for feedback. In one example, each simultaneous drive period 1321-1324 may last 8 ms (milliseconds), followed by a feedback measurement period 1331, 1341, 1332, and 1342 of 2 ms.

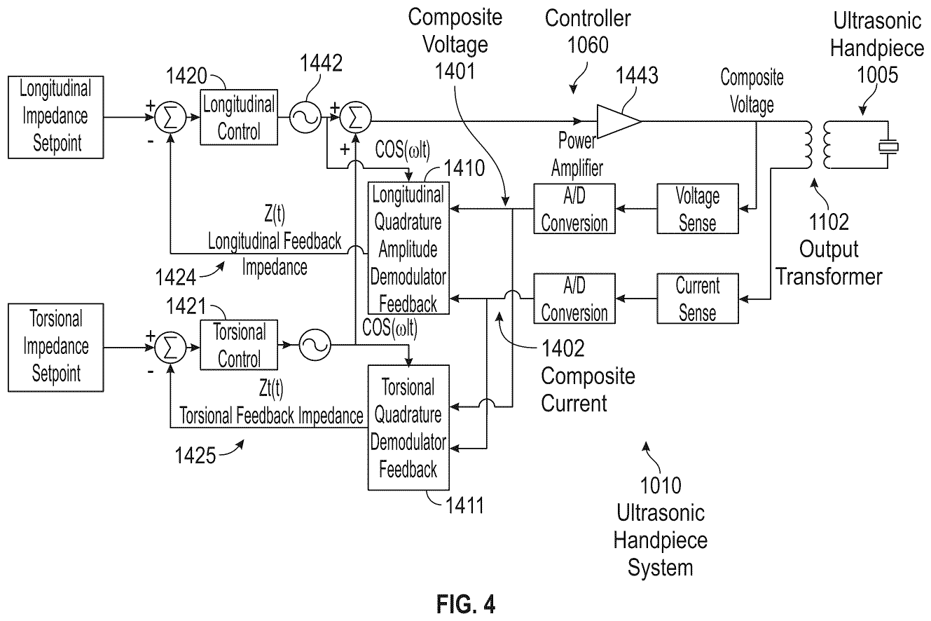

FIG. 4 is a detailed block diagram of ultrasonic handpiece system 1010 in accordance with additional example embodiments. In contrast to the embodiments of FIG. 1B and FIG. 3, where the simultaneous application of multiple modes of oscillation on a single piezoelectric element 1014 is active less than 100% of the time, e.g., approximately 80% of the time, in FIG. 4 the simultaneous application is active 100% of the time. Further in contrast to the embodiments of FIGS. 1B and 3, which samples the RMS value, in FIG. 4 the composite voltage 1401 and current 1402 are sampled such that the magnitude and phase of each of the independent modes of oscillation may be extracted. FIG. 4 includes frequency generator 1442, power amplifier 1443, longitudinal quadrature amplifier demodulator 1410 and a torsional quadrature amplifier demodulator 1411 that extract the specific oscillation mode feedback. The extracted longitudinal impedance magnitude 1424 (“Zl(t)”) and torsional impedance magnitude 1425 (“Zt(t)”) is then fed back to control loops 1420 and 1421, respectively. Frequency generator 1442 advantageously provides precise frequency generation capability for the required frequency signals for each oscillation mode. Such generated signals are then amplified by the power amplifier 1443 to attain the necessary power levels for driving the vibrational modes in the ultrasonic handpiece 4005.

The functionality of FIG. 4 continuously applies the multiple drive modes as well as feedback for each oscillation mode in a continuous and simultaneous manner as follows, where “model” is the longitudinal mode (in FIG. 4, “ωl” is the longitudinal angular frequency (Radians/second) and “ωlt” is the longitudinal angular frequency multiplied by time (seconds)) and “mode2” is the torsional mode (in FIG. 4, “ωt” is the torsional frequency (Radians/second) and “ωtt” is the torsional angular frequency multiplied by time (seconds)”):

Vₒᵤₜ=(V_{mode1}*cos(ω_{mode1}*t+Ø_{mode1})+V_{mode2}*cos(ω_{mode2}*t+Ø_{mode2})) Eqn. (1)

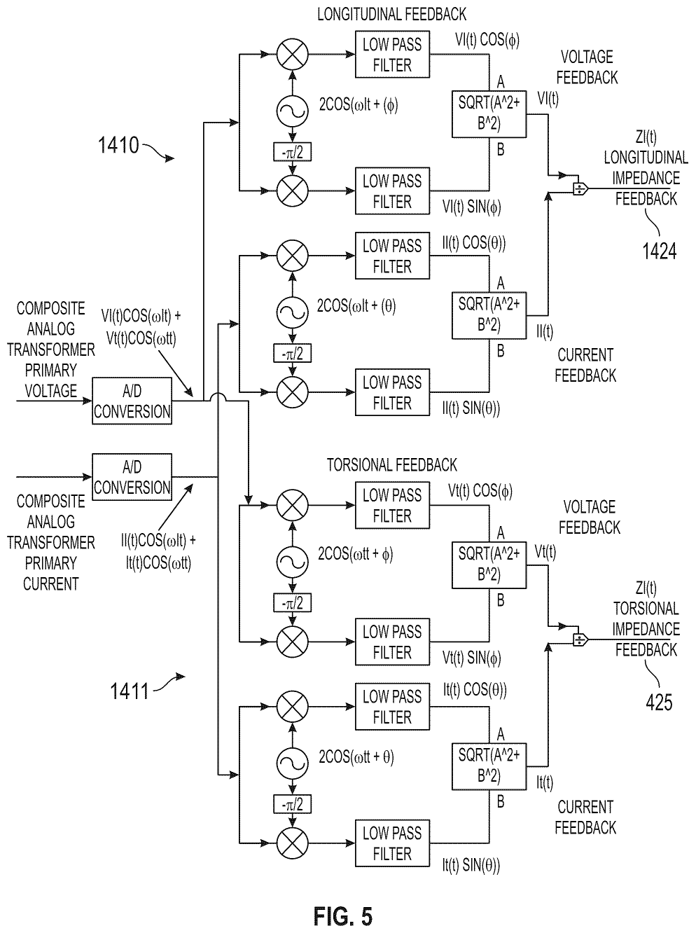

FIG. 5 is a detailed block diagram of a longitudinal quadrature amplitude demodulator 1410 and torsional quadrature amplitude demodulator 1411 that may be used in the example of FIG. 4 in accordance with some example embodiments. As shown in FIG. 5, demodulators 1410 and 1411 implement equation (1) above and provide a mixing function that isolates the sinusoidal signal produced by the longitudinal and torsional forces generated simultaneously on a single piezoelectric element 1014. In contrast, the embodiment of FIG. 1B does not digitize the entire sinusoidal signal and only digitizes the envelope of the RMS value. FIG. 5 digitizes the composite signal which is used to extract the amplitude/magnitude information of each mode of oscillation. In FIG. 5, “V” refers to voltage, “I” refers to current, “θ” refers to the current phase angle, and “ϕ” refers to the voltage phase angle.

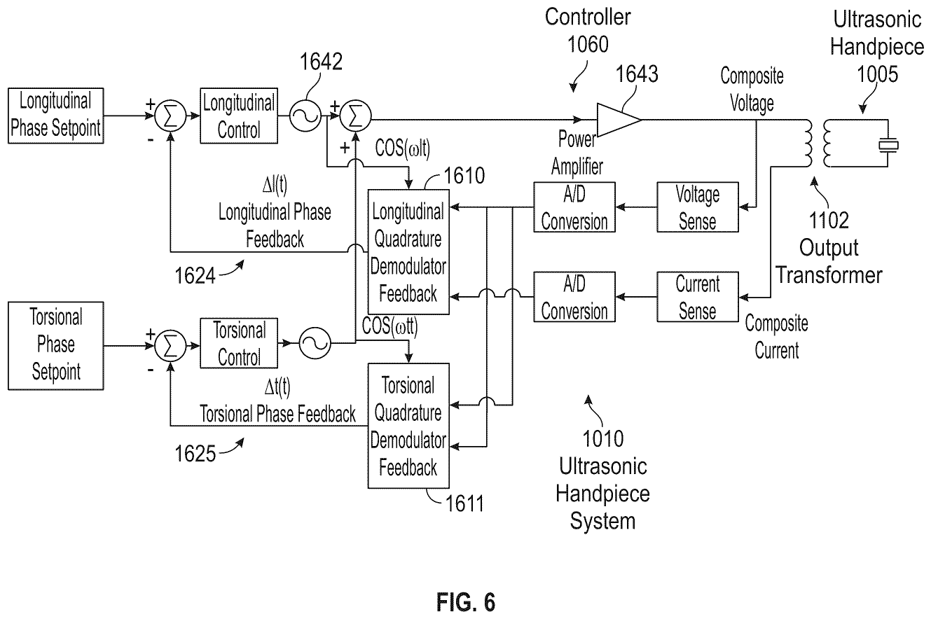

FIG. 6 is a detailed block diagram of ultrasonic handpiece system 1010 in accordance with some embodiments. The embodiment shown in FIG. 6 is similar to the embodiment of FIG. 4. However, instead of the magnitude of the impedance being used for feedback, the phase of the impedance is used. A longitudinal quadrature phase demodulator 1610 generates a longitudinal phase feedback 1624 (“Δl(t)”) and torsional quadrature phase demodulator 1611 generates a torsional phase feedback 1625 (“Δt(t)”). Frequency generator 1642, and power amplifier 1643 may operate similar to or the same as frequency generator 1442 and power amplifier 1443, described above.

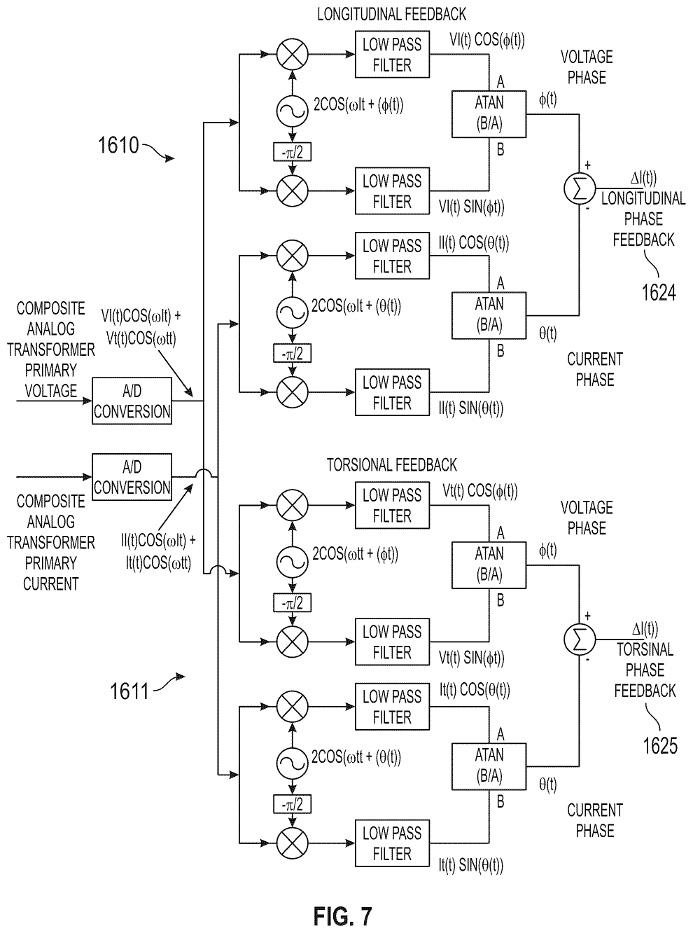

FIG. 7 is a detailed block diagram of a longitudinal quadrature phase demodulator 1610 and torsional quadrature phase demodulator 1611 that may be used in the example of FIG. 6 in accordance with some example embodiments. FIG. 7 is used to extract the phase information.

In other embodiments, a controller combines aspects of controller 1060 of FIG. 4 and controller 1060 of FIG. 6 to improve the stability of the feedback control, minimizing losses and having improved efficiency. In one embodiment, control of both amplitude/magnitude and phase are combined.

In another embodiment, both the primary and secondary side of the transformer resonant frequency are combined (in contrast to the embodiments of FIGS. 1B, 4 and 6 where only the primary side resonant frequency is monitored). Instead of feedback control to find the primary resonant frequency, embodiments include two feedback loops such that the phase is adjusted appropriately based on achieving the best secondary side frequency and the outside loop would be to drive the amplitude to achieve the primary resonant frequency. In yet another embodiment, non-linear feedback control of frequency is implemented where the controller gain and integral time are automatically adjusted according to the control error.

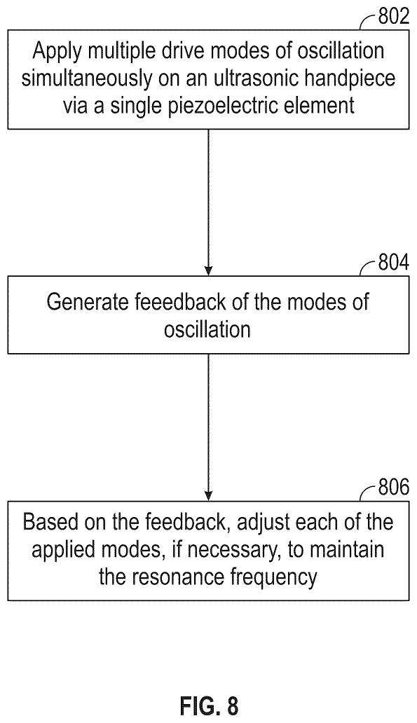

FIG. 8 is a flow diagram of an example of the functionality of controller 1060 of FIG. 1 for providing multiple modes of oscillation simultaneously for an ultrasonic handpiece 1005 via a single piezoelectric element 1014 in accordance with one embodiment. In one embodiment, the functionality of the flow diagram of FIG. 8 is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

At 802, multiple drive modes of oscillation are applied simultaneously on an ultrasonic handpiece via a single piezoelectric element. In embodiments, the multiple modes of oscillation are a torsional drive signal, e.g., of approximately 31 kHz, and a longitudinal drive signal, e.g., of approximately 45 kHz. Other frequencies can be used in other embodiments. Each mode has a resonance frequency of operation that should be maintained in order to optimize efficiency and effectiveness.

At 804, feedback of the applied modes of oscillation is generated. In one embodiment, the feedback is based on RMS values of each of the modes. In other embodiments, the feedback is based on the magnitude of the combined modes. In another embodiment, the feedback is based on the phase of the combined modes. In some embodiments, the feedback is generated in an interim period when only one of the modes is being applied. In other embodiments, the feedback is generated at the same time that both modes are being applied.

At 806, based on the feedback, each of the applied modes is adjusted, if necessary, to maintain the resonance frequency. The adjustment may include independently adjusting the frequency of the constituent components of the drive voltage so that the resulting oscillation of each mode is approximately at its resonant frequency.

Experimental results for phacoemulsification compared known systems (e.g., FIG. 2), in which a longitudinal mode and torsional mode are applied on a single piezoelectric element separately (“separate”), to embodiments of the invention in which a longitudinal mode and torsional mode are applied on a single piezoelectric element simultaneously (“simultaneous”). In the results, the average phaco time was 5.48 seconds per milligram (“s/mg”) for separate compared to 2.79 s/mg for simultaneous. The average efficiency was 6.79 joules per milligram (“j/mg”) for separate compared to 1.09 j/mg for simultaneous. The total efficiency improvement of simultaneous compared to separate is a 49.13% time reduction.

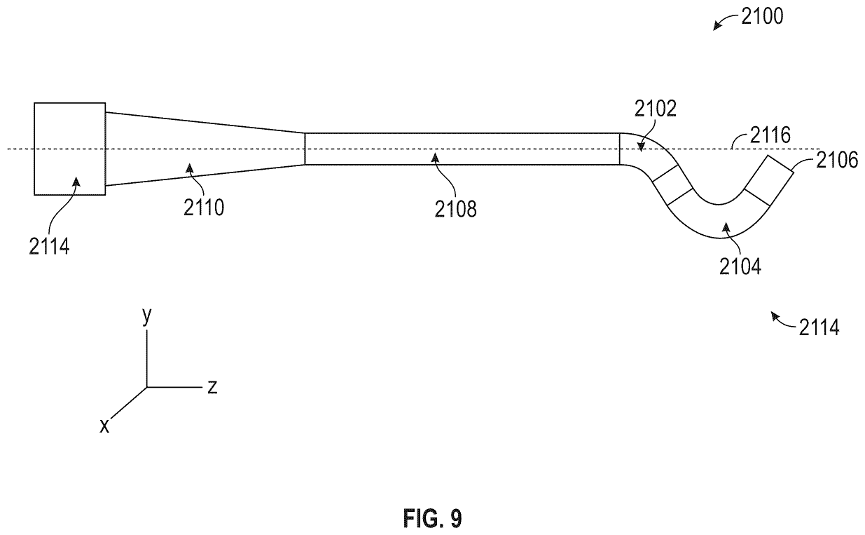

Referring now to FIG. 9 in conjunction with FIGS. 1-8, in some embodiments, working tip 1020 of system 1010 may be interchanged with a balanced tip 2100. As discussed above, an ultrasonic handpiece, such as a phacoemulsifier, operates by applying multiple modes of oscillation simultaneously using a single set of piezoelectric elements that form a piezoelectric element assembly. In some embodiments, as described in detail below, a surgical handpiece system may implement a balanced surgical tip, as described in detail below.

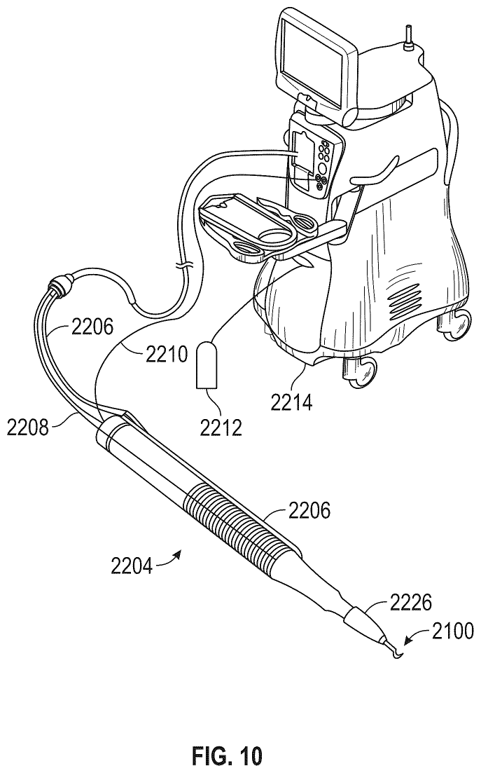

FIG. 9 illustrates a phacoemulsification balanced tip 2100 with a proximal end 2114 and a cutting edge portion 2112 that is bent relative to a shaft 2108. The balanced tip 2100 may include a predominantly straight shaft 2108 and at least two bends (first bend 2102 and second bend 2104) in the cutting edge portion 2112. Other numbers of bends are also contemplated (e.g., 3 bends, 4 bends, 7 bends, etc.). The balanced tip 2100 may be used in conjunction with a phacoemulsification handpiece 2204 (e.g., see FIG. 10). When used with the handpiece 2204, the balanced tip 2100 may be vibrated longitudinally and/or torsionally, i.e., by rotating the tip 2100 back and forth around its axis. The bends 2102/2104 may be positioned, for example, along approximately the distal 5 to 25% of the length of the balanced tip 2100) (which may be a portion positioned approximately 5 mm from the distal end point 2106 of the tip (measured along the axis 2116) through to approximately 12 mm from the distal end point 2106 of the tip 2100). Other portions of the length are also contemplated.

In some embodiments, balanced tip 2100 may include a geometry (e.g., the geometry of a conical portion 2110 of the tip, the shaft 2108, and/or the at least first bend 2102 and second bend 2104) such that, during ultrasonic torsional vibration of balanced tip 2100, a lateral displacement of the shaft 2108, perpendicular to the shaft 2108, along its length may be less than approximately 5% to 25% (e.g., 15%) of the lateral displacement of the distal end point 2106 of balanced tip 2100 (e.g., as measured during frequencies the tip is vibrated at during an ophthalmic procedure). Other thresholds may also be used (e.g., 10 to 20%, 15 to 30%, 10 to 40%, etc.). In some embodiments, lateral displacement of the tip (during expected operational frequencies) at the distal end point 2106 may be approximately in a range of 30 to 200 microns. As an example, if the distal end point 2106 has a lateral displacement of approximately +/−0.035 mm during ultrasonic torsional vibration of balanced tip 2100, the geometry of the tip may be configured such that the maximum lateral displacement along the shaft is less than 5 microns (other displacements are also possible). As another example, if the distal end point 2106 has a lateral displacement of approximately +/−0.055 mm during ultrasonic torsional vibration of the tip 2100, the geometry of the tip may be configured such that the maximum lateral displacement along the shaft is less than 8 microns. In some embodiments, the shaft may extend from the end of a conical portion 2110 (which may be, for example, approximately 12 mm from the distal end point 2106) through to the first bend 2102 in the cutting edge portion 2112 (which may be, for example, approximately 5 mm from the distal end point 2106). Other locations of the first bend 2102 are also contemplated (e.g., 3 mm, 8 mm, etc. from the distal end point 2106).

FIG. 10 illustrates a phacoemulsification surgical console 2214 connected to a handpiece 2204 through an irrigation line 2206 and an aspiration line 2208. In some embodiments, power may be supplied to handpiece 2204 through electrical cable 2210 and flow through irrigation/aspiration lines 2206 and 2208 may be controlled by a user (e.g., via footswitch 2212) to perform a phacoemulsification procedure. System 1010 may similarly be employed on surgical console 2214.

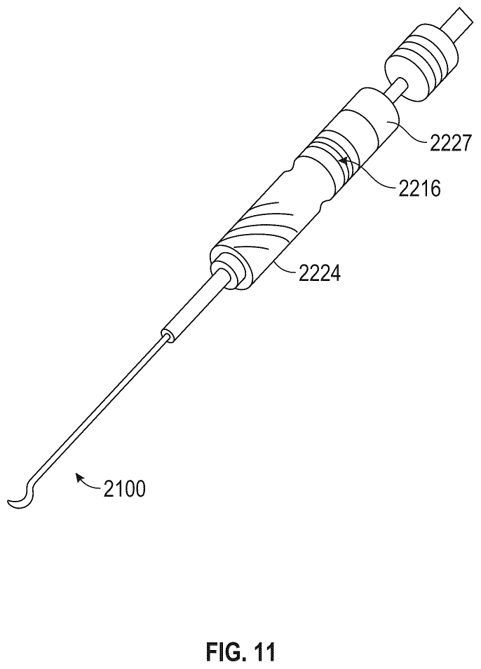

As shown in FIG. 11, in some embodiments, handpiece 2204 may be coupled to a phacoemulsification balanced tip 2100. In some embodiments, the handpiece 2204 may include at least one set of piezoelectric elements 2227 polarized to produce longitudinal motion when excited at a relevant resonant frequency. As seen in FIG. 3, the piezoelectric elements 2227 may be connected to an ultrasonic horn 2216 to which a balanced tip 2100 is attached. The horn 2216 and/or the balanced tip 2100 may include a plurality of diagonal slits or grooves 2224. The slits or grooves 2224 may produce torsional movement in the balanced tip 2100 when the piezoelectric crystals are excited at a resonant frequency. Movement of the balanced tip 2100 caused by the grooves 2224 engaging fixed elements in the handpiece 2204 may include a torsional rotational component relative to a centerline of the horn 2216.

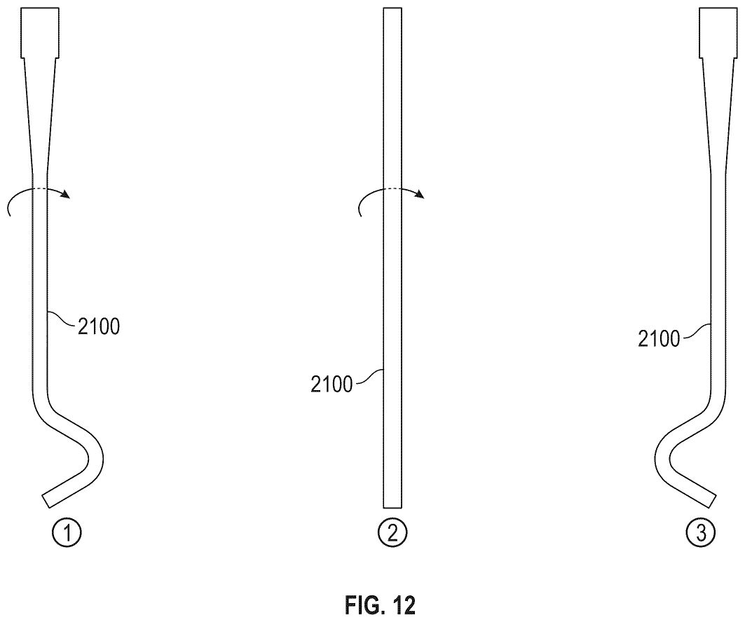

As shown in FIG. 12, in some embodiments, the balanced tip 2100 may be configured for ultrasonic torsional rotation back and forth through approximately an arc in the range of approximately 2 to 6 degrees (e.g., an arc of 4 degrees). Other arcs are also contemplated (e.g., 10 degree arc (e.g., plus or minus 5 degrees off center (see middle diagram 2), plus or minus 20 degrees off center, plus or minus 90 degrees off center, etc.)). In some embodiments, the balanced tip 2100 may be ultrasonically torsionally vibrated at a frequency of approximately between 10-60 kHz (e.g., 31 kHz). Other arcs and frequencies are also contemplated. For example, an arc of plus or minus 20 degrees and/or a frequency of 42 kHz may be used. The arc shown in FIG. 12 is exaggerated to show movement (i.e., the total arc shown is 180 degrees, whereas the balanced tip 2100 may have an arc of 4 degrees). In some embodiments, the tip movement in FIG. 12 may also include a longitudinal component (e.g., up and down along an axis parallel to the shaft).

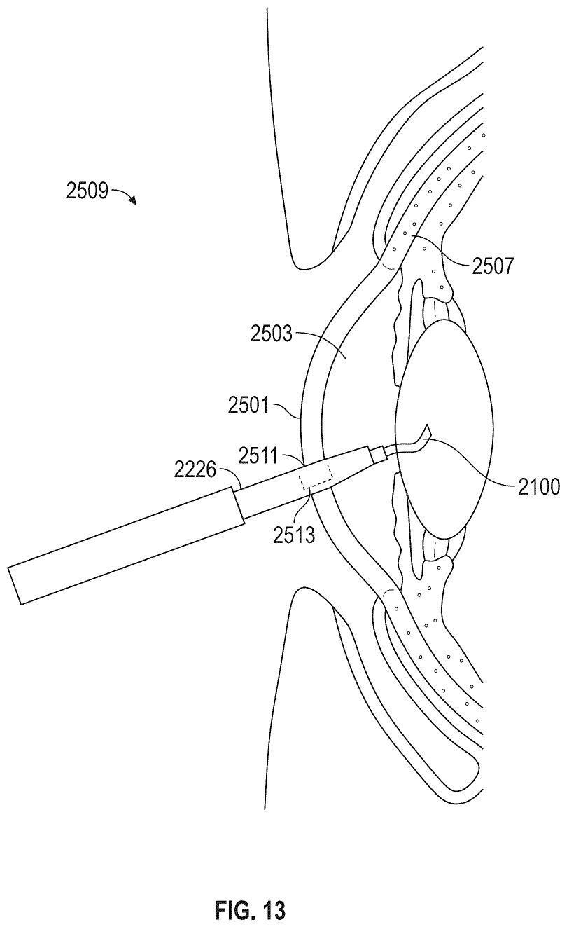

As seen in FIG. 13, when used to perform phacoemulsification, the ends of the balanced tip 2100 and an irrigating sleeve 2226 may be inserted into a small incision 2511 in the cornea 2501, sclera 2507, or other location in the eye tissue to gain access to, for example, the anterior chamber 2503 of the eye 2509. In various embodiments, a portion or all of the balanced tip 2100 may be inside the irrigating sleeve 2226. A portion 2513 of balanced tip 2100 along the incision 2511 may be in thermal contact with the incision 2511 (and/or other parts of the eye) through the irrigating sleeve 2226 during the phacoemulsification procedure. In some embodiments, the portion 2513 along the incision 2511 may be in direct contact with the incision 2511 (e.g., in the absence of the sleeve 2226). The balanced tip 2100 may be ultrasonically torsionally vibrated along its longitudinal axis within the irrigating sleeve 2226 by a crystal-driven ultrasonic horn 2216, thereby emulsifying upon contact the selected tissue in situ. The hollow bore of the balanced tip 2100 may communicate with the bore in the horn that in tum may communicate with the aspiration line from the handpiece 2204 to the console 2214. A reduced pressure or vacuum source in the console 2214 may draw or aspirate the emulsified tissue from the eye 2509 through an open end of the balanced tip 2100, the bore of the balanced tip 2100, the horn bore, and the aspiration line 2208 and into a collection device. The aspiration of emulsified tissue may be aided by a saline flushing solution or irrigant that may be injected into the surgical site through the small annular gap between the inside surface of the irrigating sleeve 2226 and an outside surface of the balanced tip 2100.

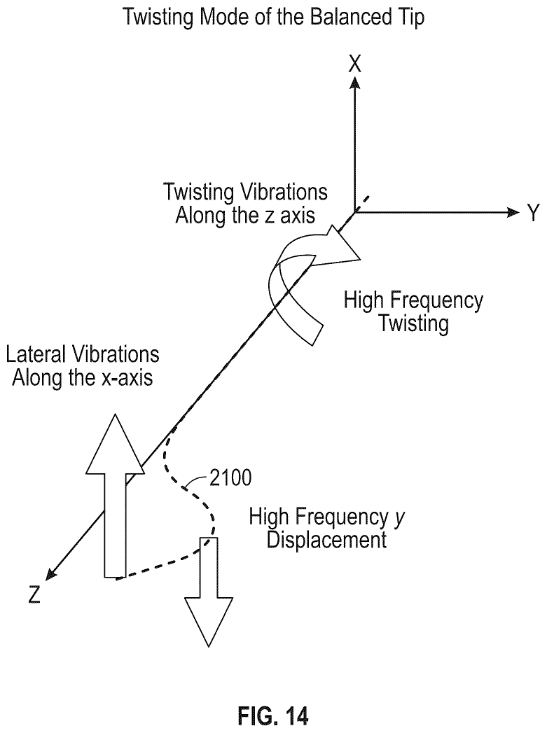

As seen in FIG. 14, ultrasonic torsional vibrations of the balanced tip 2100 may result in at least two motions: 1) lateral displacement, of the balanced tip 2100 from its equilibrium position, perpendicular to an axis 2116 that is collinear with a straight shaft portion (axis 116 may be denoted as the “z-axis”) and perpendicular to an axis of a bend of the tip (denoted as y-axis in FIG. 1) (the y-axis and z-axis forming a plane that includes the bend); 2) twist angle along the z axis of the balanced tip 2100. An unbalanced tip may have significant bending along the tip length (especially in the shaft) under the action of torsional vibration. By balancing the tip 2100 as described herein, lateral displacement along the shaft of balanced tip 2100 may be reduced while the lateral displacement at the distal end point 2106 of balanced tip 2100 may be increased. Twisting vibrations may be present in balanced tip 2100 (e.g., twisting back and forth along a twist angle relative to the z axis) which may lead to a relatively large lateral displacement of the distal end point 2106 in addition to or in the absence of the lateral displacement of the shaft 2108.

In some embodiments, balancing tip 2100 may include adjusting the tip geometry and physically testing a tip with the adjusted tip geometry or using modeling equations or Finite Element Analysis (FEA) to model tip vibrations to find a tip geometry that results in reduced lateral displacement along the shaft 2108 with increased lateral displacement and twisting at the distal end point 2106 (e.g., using software such as ANSYS). Tip geometry characteristics may include, for example, number of bends (e.g., bends 2102, 2103), location of the bends, length of the shaft, diameter of the shaft 2108, length of the conical portion 2110, and diameter of the conical portion. Other tip geometry characteristics may also be modified. In some embodiments, different tip geometries may be tested, for example, by physically creating tips with various tip geometries, vibrating the tips (e.g., using frequencies and modes that are used during phacoemulsification) and monitoring lateral displacement and/or heat generated by the various tip geometries. One or more iterations of testing different tip geometries (e.g., by fixing the location of one bend in the tip and testing different tips with different second bend locations and curvature) may result in identifying one or more optimized tip geometries. Other numbers of bends and geometric modifications (e.g., modifying the location of both bends while holding curvature of both bends constant, modifying the location and curvature of the bends, modifying the number of bends, modifying the length of the shaft, modifying the length of the conical portion, modifying the radius of the shaft, modifying the radii of the conical portion, etc.) are also possible.

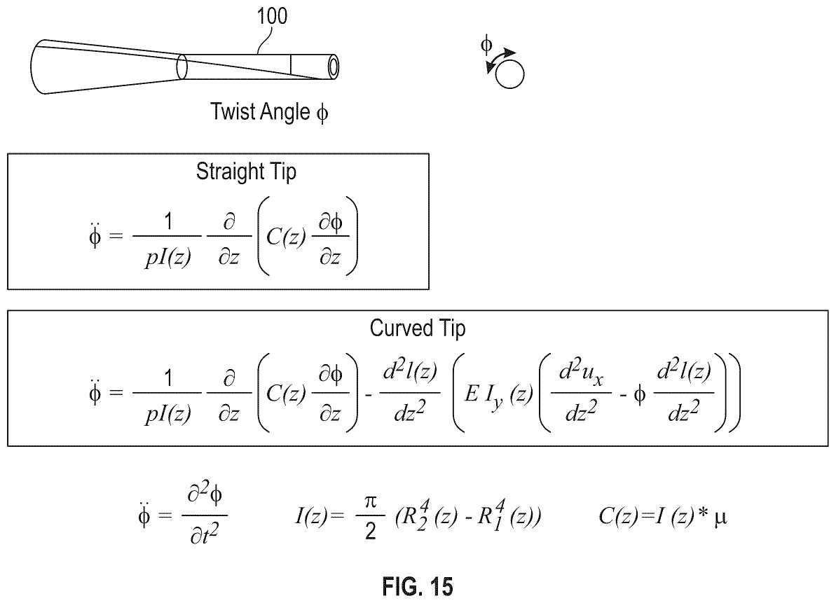

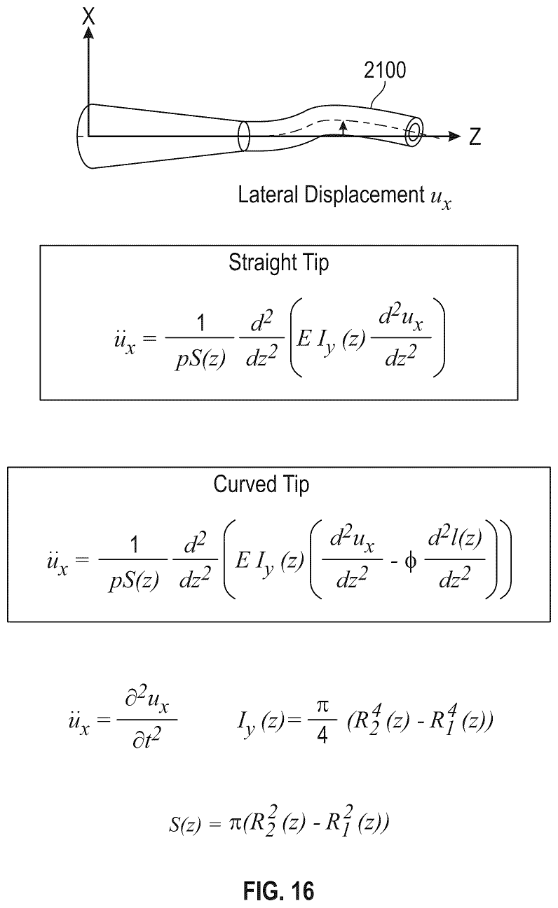

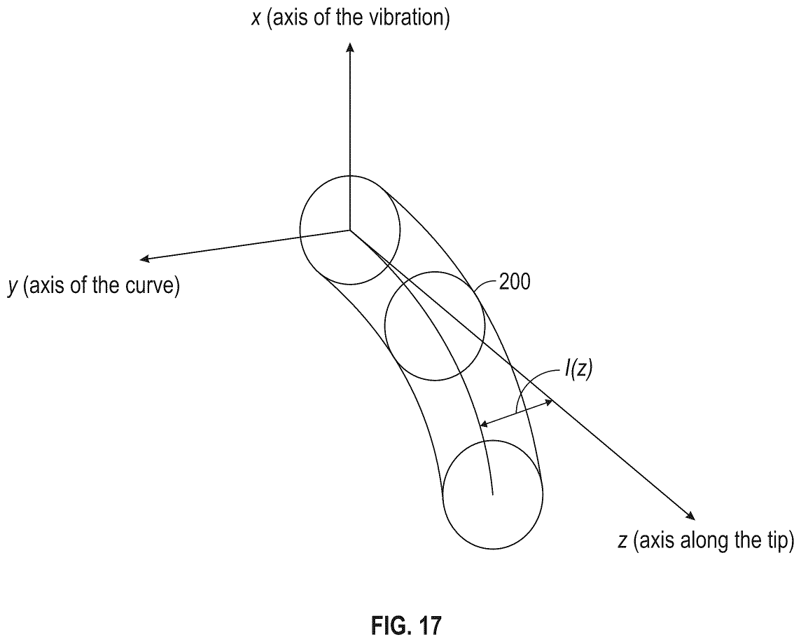

In some embodiments, modeling equations may be used (in place of or in addition to FEA and/or physical testing) to test different tip geometries. For example, the equations describing how the twist angle (ϕ) and the lateral displacement (uₓ) vary along the z axis while ultrasonically torsionally vibrating a straight (predominantly cylindrical) tip (e.g., based on the general elasticity theory) may be represented as follows (see also FIGS. 15-16):

$$\ddot{ϕ} = \frac{1}{ρ I ( z )} \frac{\partial }{\partial z} ( C ( z ) \frac{\partial ϕ}{\partial z} ) where$$

$$\ddot{ϕ} = \frac{{\partial }^{2} ϕ}{\partial {t}^{2}} ; I ( z ) = \frac{π}{2} ( {R}_{2}^{4} ( z ) - {R}_{1}^{4} ( z ) ) ; C ( z ) = I ( z ) * μ$$

$${\ddot{u}}_{x} = \frac{1}{ρ S ( z )} \frac{{d}^{2}}{{dz}^{2}} ( {EI}_{y} ( z ) \frac{{d}^{2} {u}_{x}}{{dz}^{2}} ) where$$

$${\ddot{u}}_{x} = \frac{{\partial }^{2} {u}_{x}}{\partial {t}^{2}} ; {I}_{y} ( z ) = \frac{π}{4} ( {R}_{2}^{4} ( z ) - {R}_{1}^{4} ( z ) ) ;$$

$$and S ( z ) = π ( {R}_{2}^{2} ( z ) - {R}_{1}^{2} ( z ) )$$

Where ϕ is the twist angle of the tip, p is density of the tip material, I(z) is the moment of inertia of the cylindrical tip cross-section around the z axis, R₁(z) is the inner radius of a hollow inner section of the cylindrical tip body (if the cylindrical body is solid, R₁(z) may be 0 along the entire z axis); R₂(z) is the outer radius of a cylindrical tip body; t is time, uₓ is lateral displacement along the x-axis, S(z) is the cross-sectional area of the cylindrical tip along the z axis, E is young's modulus of the tip material, I_{y}(z) is the moment of inertia of the cross-section of a cylindrical tip around the y axis, and μ is the torsional modulus of the tip material. Characteristics such as p may be the same for the entire tip while characteristics such as R₁(z) and R₂(z) may vary along the z-axis (and thus may be represented, for example, as an array of values). The equations describing how the twist angle (ϕ) and the lateral displacement (uₓ) vary along the z axis while ultrasonically torsionally vibrating a curved (predominantly cylindrical) tip (e.g., a tip with bends 102/103) may be represented as follows (see also FIGS. 15-16):

$$\ddot{ϕ} = \frac{1}{ρ I ( z )} \frac{\partial }{\partial z} ( C ( z ) \frac{\partial ϕ}{\partial z} ) - \frac{{d}^{2} l ( z )}{{dz}^{2}} ( {EI}_{y} ( z ) ( \frac{{d}^{2} {u}_{x}}{{dz}^{2}} - ϕ \frac{{d}^{2} l ( z )}{{dz}^{2}} ) )$$

$$where$$

$$\ddot{ϕ} = \frac{{\partial }^{2} ϕ}{\partial {t}^{2}} ; I ( z ) = \frac{π}{2} ( {R}_{2}^{4} ( z ) - {R}_{1}^{4} ( z ) ) ; C ( z ) - I ( z ) * μ$$

$${\ddot{u}}_{x} = \frac{1}{ρ S ( z )} \frac{{d}^{2}}{{dz}^{2}} ( {EI}_{y} ( z ) ( \frac{{d}^{2} {u}_{x}}{{dz}^{2}} - ϕ \frac{{d}^{2} l ( z )}{{dz}^{2}} ) ) where$$

$${\ddot{u}}_{x} = \frac{{\partial }^{2} {u}_{x}}{\partial {t}^{2}} ; {I}_{y} ( z ) = \frac{π}{4} ( {R}_{2}^{4} ( z ) - {R}_{1}^{4} ( z ) ) ;$$

$$and S ( z ) = π ( {R}_{2}^{2} ( z ) - {R}_{1}^{2} ( z ) )$$

Where ϕ is the twist angle of the tip, ρ is density of the tip material, I(z) is the moment of inertia of the cylindrical tip cross-section around the z axis, R₁(z) is the inner radius of a hollow inner section of the cylindrical tip body (if the cylindrical body is solid, R₁(z) may be O); R₂(z) is the outer radius of a cylindrical body; t is time, uₓ is lateral displacement along the x-axis, S(z) is the cross-sectional area of the cylindrical tip along the z axis, E is young's modulus of the tip material, I_{y}(z) is the moment of inertia of the cross-section of a cylindrical tip around the y axis, μ is the torsional modulus of the tip material, and l(z) is lateral displacement along the y axis as seen in FIG. 17. In some embodiments, one or more of the inputs and/or equations may be modified to account for the presence of a medium the tip is vibrating in (e.g., water, vitreous, etc.). For example, the equation for lateral displacement of the tip may be modified as follows:

$${\ddot{u}}_{x} - γ {\dot{u}}_{x} = \frac{1}{ρ S ( z ) + {ρ}_{Media} {S}_{Media} ( z )} \frac{{d}^{2}}{{dz}^{2}} ( {EI}_{y} ( z ) ( \frac{{d}^{2} {u}_{x}}{{dz}^{2}} - ϕ \frac{{d}^{2} l ( z )}{{dz}^{2}} ) )$$

Where γ is an empirical parameter that represents dissipation due to media (such as water). The value of γ may be adjusted to align the equation with measured displacements of existing tips in the media. The ρ_{Media}S_{Media}(z) term in the denominator represents the increase of the tip mass due to the media that is following the motion of the tip. The ρ_{Media} term is the density of media and the S_{Media}(z) is the cross section of the media moving together with the tip, which may be evaluated using ideal fluid theory as: S_{Media}(z)=π(R₁²(z)+R₂²(z)) (where R₁ is the inner diameter of the media mass and R₂ is the outer diameter of the media mass following the tip). Other modifications are also contemplated.

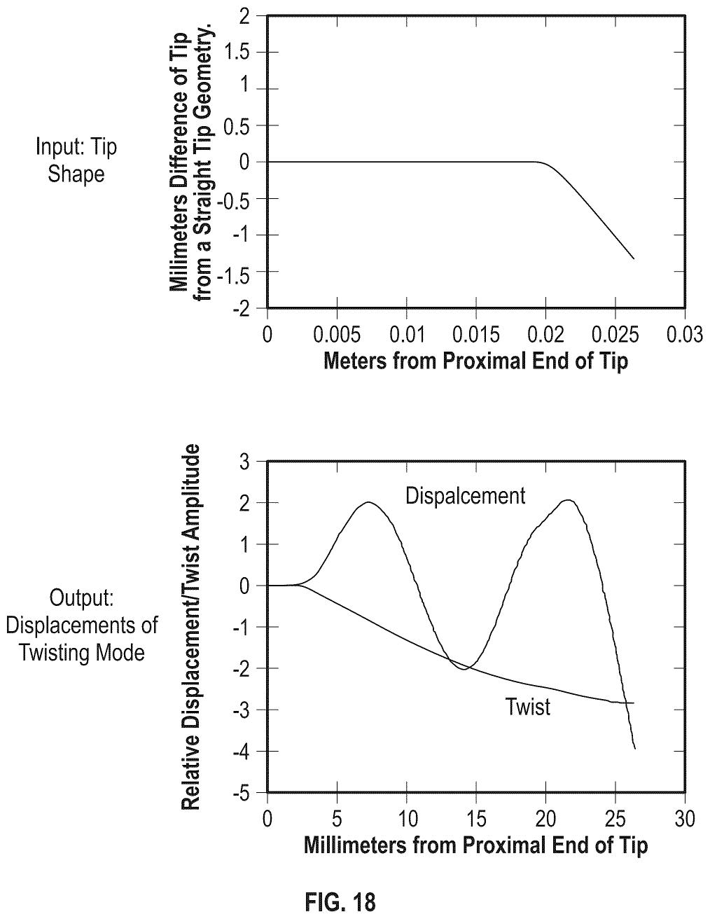

In some embodiments, along with the various tip characteristics (e.g., ρ, E, etc.), geometric characteristics (e.g., S(z), I(z), C(z), I_{y}(z), etc.) may be entered by a user or computed by modeling software (e.g., MATLAB™) based on other inputs provided by the user (e.g., the user may provide an inner radius (if the tip is hollow in the center), an outer radius of the tip along the z axis, a location (e.g., starting and stopping points (along the z axis) and curvature of one or more bends, etc.). The user may also draw the tip shape using a graphical user interface (e.g., see input plots in FIG. 18), the user may preload a tip geometry (e.g., a three dimensional rendering), etc. In some embodiments, the outer radius may be large at values of small z (i.e., in the conical portion of the tip) and relatively small at the end of the tip. Other inputs are also contemplated.

In some embodiments, the solutions for ϕ and uₓ from the equations above may be used to examine the lateral displacement and twist angles along the z axis for different tip geometries and a balanced/tuned tip geometry may be selected from several tip geometries that maximizes the lateral displacement uₓ, and twist angle ϕ of the distal end point 106 while minimizing the lateral displacement uₓ along the tip length (e.g., along the shaft 108). In some embodiments, solving for ϕ and uₓ may include using harmonic analysis. A solution of the equations for ϕ and uₓ may provide the twist angle and/or lateral displacements as functions of both z and t (e.g., u(z,t) and ϕ(z,t)). These solutions may then be used to model the tip according to a harmonic force. Modeling according to a harmonic force may include modeling the tip as if the tip oscillates at some frequency ω like cos(ωt). Harmonics may thus be used to simplify the modeling equations for u(z,t) and ϕ(z,t) according to the equations for \ddot{ϕ} and üₓ, provided above. In some embodiments, the solution may be modeled according to u(z)cos(ωt) (i.e., the vibrational amplitude may be modeled to depend only on z). The formula u(z)cos(ωt) may be used in the equations of motion (\ddot{ϕ} and üₓ) to provide a differential equation for the amplitude of vibrations u(z) that is independent of time. The solutions for tip displacement amplitude and twist amplitude may then be plotted (e.g., see outputs in FIG. 18). In some embodiments, harmonic analysis may not be used (e.g., various solutions dependent on time and z may be determined and analyzed).

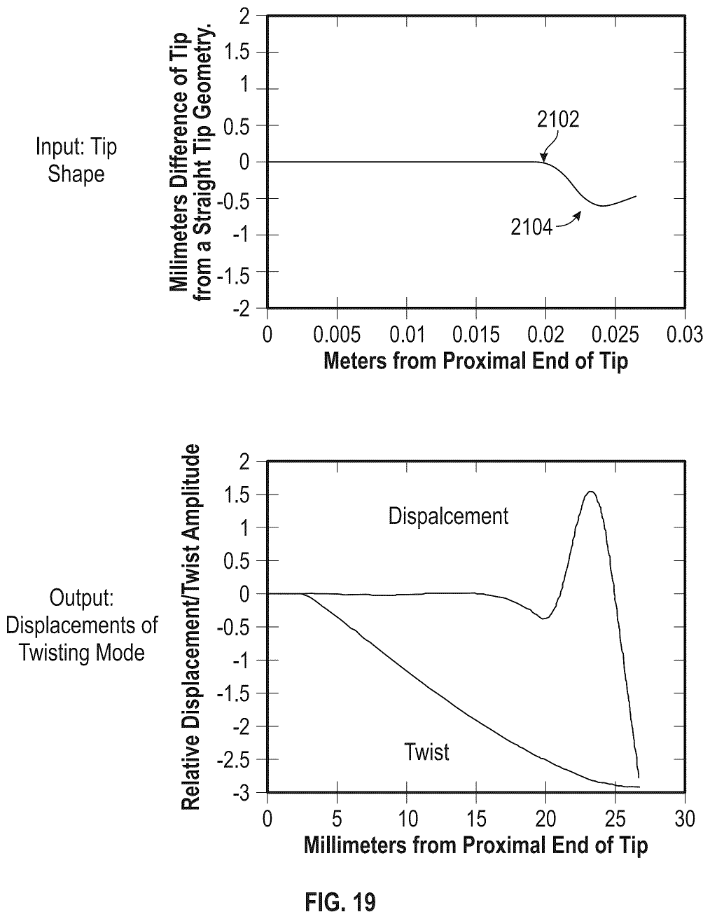

Referring now to FIGS. 18-19, FIGS. 18-19 illustrate input tip shapes and the corresponding displacement and twist angles along the tip length according to the model equations provided above. In some embodiments, the position and the curvature of the first bend 2102 may be selected based on various factors such as ergonomics and manufacturing considerations. The second bend 2104 may be positioned closer to the cutting edge portion 2112 of the balanced tip 2100. The curvature of this bend may then be chosen using the prediction of the model equations provided above. The resulting tip shape may then be verified and/or adjusted by performing finite element analysis simulations. The ideal curvature may be such that the twisting vibrational mode and the bending vibrational mode of the balanced tip 2100 are uncoupled. The motion of the balanced tip 2100 under the torsional force may be the same as its twisting vibrational mode. In some embodiments, the tip bends (e.g., 2102/2103) may be positioned such that the ultrasonic torsional vibration energy in the balanced tip 2100 may be in a twisting vibrational mode along a substantial portion of the shaft 2108 (with reduced lateral motion). In some embodiments, the length of the shaft 2108 may also be adjusted to tune the twisting vibrational mode such that that twisting vibration is in resonance with the ultrasonic driving mechanism (e.g., the piezoelectric elements 2227 in the handpiece combined with a horn) to increase twisting displacement at the distal end point 2106. In some embodiments, the amplitude of the distal end point lateral displacement of the balanced tip 2100 may depend on the resonance between the torsional driving force and the twisting vibrational mode. While the driving frequency may be set by the torsional horn design, the frequency of the twisting mode may be adjusted by selecting, for example, a length of the conical portion 2110 of the balanced tip 2100. The length of the conical portion 2110 may be chosen to maximize the twisting vibrations of the balanced tip 2100 thus resulting in the maximum twisting displacement of the distal end point 2106. Other tip characteristics may also be varied.

In some embodiments, L-Motion may be implemented as an additional mode of vibrations. For example, so-called L-motion may be facilitated combining the driving forces of torsional and longitudinal vibrations, resulting in a motion of the phacoemulsification tip. So-called “L-motion” is characterized by a Lissajous function (i.e., L-function). Such motion represents the fusion of torsional and longitudinal vibrations, creating a unique Lissajous pattern in the phacoemulsification tip's motion. It has been determined that such pattern provides unexpected results stemming from this particular shape, which has demonstrated remarkable performance in breaking apart hard materials inside the eye, revolutionizing the approach to cataract surgeries and other challenging ocular procedures.

The L-motion, enabled by the integration of torsional and longitudinal vibrations in the surgical handpiece, has proven to be highly effective in addressing dense and resistant cataractous lens material. The Lissajous function employed in the L-motion produces an intricate pattern of vibrational motion in the phacoemulsification tip. Such Lissajous pattern exhibits harmonious oscillations along both the torsional and longitudinal axes, offering a balanced combination of cutting efficiency and precise aspiration. The tip's motion substantially aligns with the Lissajous pattern, contributing to smoother and more effective lens fragmentation and removal.

The interplay between torsional and longitudinal vibrations in generating the L-motion brings a new dimension of versatility and adaptability to the surgical handpiece. The L-motion's ability to precisely target and break apart hard materials inside the eye reduces the need for excessive energy application, mitigating the risk of potential thermal injury and post-operative complications.

Furthermore, the unexpected performance of the L-motion extends beyond its cataract-specific applications. The motion's intricate shape and motion dynamics have shown potential benefits in addressing other hard materials within the eye, such as residual lens fragments or calcified deposits. The L-motion's versatility in handling various ocular challenges marks a significant advancement in ocular surgery, contributing to improved patient outcomes and elevating the standards of surgical care.

In some embodiments, the balanced tip 2100 may have a diameter in a range of approximately 0.5 mm to 2 mm (e.g., 1.5 mm). In some embodiments, the balanced tip 2100 may have a diameter at a top of the tip of approximately 1.5 mm and a diameter near a distal end of the tip of 0.9 mm (other diameters and configurations are also contemplated). In one embodiment, the balanced tip 2100 may have a length of approximately 1⅜ inches and the bends 2102, 2103 may be located along the distal approximate ⅛ and 2/8 inches. Other dimensions are also contemplated. In some embodiments the first bend 2102 may be approximately in a range of −10 to −30 degrees while the second bend 2104 may be approximately in a range of 20 to 50 degrees. Other bend angles are also contemplated. The cutting edge portion 2112 may have a flared, tapered and/or beveled end (in some embodiments, the cutting edge portion 2112 may be flat). Balanced tip 2100 may be made from stainless steel or titanium (other materials may also be used). Balanced tip 2100 may have an overall length of between 0.50 inches and 1.50 inches (e.g., 1.20 inches). Other lengths are also contemplated. Balanced tip 2100 may be formed using conventional metalworking technology and may be electro polished. Shaft 2108 may be generally tubular, with an outside diameter of between 0.005 inches and 0.100 inches and an inside diameter of between 0.001 inches and 0.090 inches (other diameters are also contemplated).

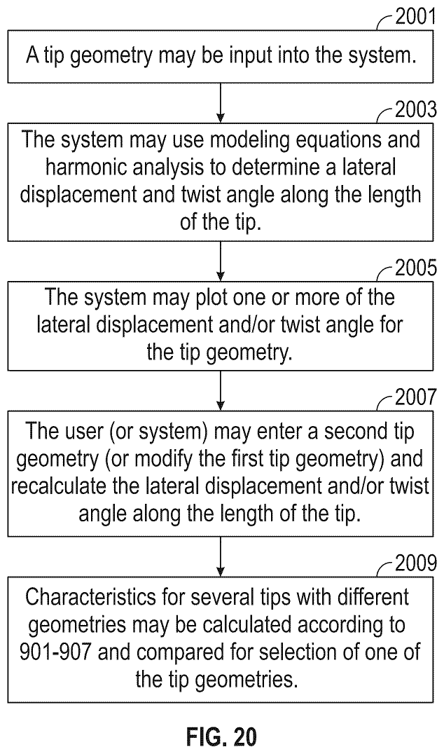

FIG. 20 illustrates a flowchart of the method for determining a tip geometry, according to an embodiment. The elements provided in the flowchart are illustrative only. Various provided elements may be omitted, additional elements may be added, and/or various elements may be performed in a different order than provided below.

At 2001, a tip geometry may be input into the system. For example, geometry inputs may be stored in an input file. In some embodiments, the tip geometry may include one or more of the following defined as values of the geometry at slices of the tip (e.g., the tip may be divided into 500 slices and the geometric characteristics of the tip at each slice may be stored in a separate array assigned to a respective geometric variable). For example, geometric characteristics for the tip slices may include curvature (e.g., in degrees), torsional rigidity (e.g., C(z)), moment of inertia around the x-axis (e.g., I(z)), cross sectional area (e.g., S(z)), moment of inertia of the slice around the y-axis that controls the bending rigidity of the tip (e.g., Iy(z)), distance of the tip from the z-axis (e.g., l(z)). Other inputs are also contemplated.

In some embodiments, these slice based arrays may be input directly by a user or may be calculated based on other geometric inputs. For example, the user may provide a length of the tip, the length of the conical portion, the location along the tip where the first bend starts, the location along the tip where the first bend ends, the curvature of the first bend, the location along the tip where the second bend starts, the location along the tip where the second bend ends, the curvature of the second bend, the shear modulus of the tip material, young's modulus for the tip material, the density of the tip material, etc. and the specific inputs for the different slices may be calculated and stored in an input file or provided to modeling software. In some embodiments, the computer system may generate the inputs automatically. For example, the computer system may cycle through various iterations of possible tip geometries. In some embodiments, the user may draw a tip (e.g., through a graphical user interface) and the computer system may calculate the geometry based on the drawing. Other input types are also contemplated.

At 2003, the system may use modeling equations and harmonic analysis to determine a lateral displacement and twist angle along the length of the tip for the given tip geometry and ultrasonic torsional vibration frequency (e.g., approximately 31 kHz). Other frequencies are also contemplated. For example, the equations below:

$${\ddot{u}}_{x} = \frac{1}{ρ S ( z )} \frac{{d}^{2}}{{dz}^{2}} ( {EI}_{y} ( z ) ( \frac{{d}^{2} {u}_{x}}{{dz}^{2}} - ϕ \frac{{d}^{2} l ( z )}{{dz}^{2}} ) ) and$$

$$\ddot{ϕ} = \frac{1}{ρ I ( z )} \frac{\partial }{\partial z} ( C ( z ) \frac{\partial ϕ}{\partial z} ) - \frac{{d}^{2} l ( z )}{{dz}^{2}} ( {EI}_{y} ( z ) ( \frac{{d}^{2} {u}_{x}}{{dz}^{2}} - ϕ \frac{{d}^{2} l ( z )}{{dz}^{2}} ) )$$

may be solved for uₓ and ϕ using inputs (as defined above) and harmonic analysis (e.g., using harmonics with u(z)cos(ωt), ϕ(z)cos(ωt)) to simplify the results by removing time. According to harmonic analysis, in some embodiments, it may be assumed that: uₓ(z,t)=u(z)cos(ωt), and ϕ(z,t)=φ(z)cos(ωt)

By taking a time derivative:

$${cos ( ω t )}^{″} = - {ω}^{2} cos ( ω t )$$

and substituting the time derivative into the original equations and cancelling the time cosine one may obtain time independent equations:

$$- {ω}^{2} u ( z ) = \frac{1}{ρ S ( z )} \frac{{d}^{2}}{{dz}^{2}} ( {EI}_{y} ( z ) ( \frac{{d}^{2} u ( z )}{{dz}^{2}} - φ ( z ) \frac{{d}^{2} l ( z )}{{dz}^{2}} ) ) and$$

$$- {ω}^{2} φ ( z ) = \frac{1}{ρ I ( z )} \frac{\partial }{\partial z} ( C ( z ) \frac{\partial φ ( z )}{\partial z} ) - \frac{{d}^{2} l ( z )}{{dz}^{2}} ( {EI}_{y} ( z ) ( \frac{{d}^{2} u ( z )}{{dz}^{2}} - φ ( z ) \frac{{d}^{2} l ( z )}{{dz}^{2}} ) )$$

The above equations may then solved for the amplitudes of displacement u(z) and the twist angle φ(z). Other equations for solving uₓ may also be used.

At 2005, the system may plot one or more of the lateral displacement and/or twist angle for the tip geometry (e.g., see FIGS. 16-17). At 2007, the user (or system) may provide a second tip geometry (or modify the first tip geometry) and recalculate the lateral displacement (uₓ) and twist angle (ϕ) along the length of the tip. Other modeling techniques may also be used. For example, finite element analysis (FEA) may be used to determine lateral displacement (uₓ) and/or twist angle (ϕ) along the length of tips of various geometries subjected to various vibrations. Further, other equations may also be used (e.g., different equations may be used for square tip modeling).

At 2009, characteristics for several tips with different geometries may be calculated according to 2001-2007 and compared for selection of one of the tip geometries (or generation of a new tip geometry to analyze). Selecting one of the tip geometries may include selecting a tip geometry based on which tip geometry has a smaller lateral displacement along a portion of the tip shaft configured to be along an incision in an eye during a phacoemulsification procedure. In some embodiments, if the lateral displacement (of the analyzed tip geometries) along a portion of the tip shaft configured to be along an incision in an eye during a phacoemulsification procedure (e.g., throughout a portion of the shaft that extends from the proximal end of the shaft (such as the end of the conical portion) to the first bend of the cutting edge portion) is greater than approximately 5% to 25% (e.g., 15%) of the lateral displacement of the distal end point 2106, (other thresholds (e.g., 1 micron, 2 microns, 100 microns, 2 mm, etc.) may also be used), another tip geometry may be generated, the lateral displacement of the new tip geometry may be modeled and compared to at least one of the lateral displacement of the first or second tip geometry for further selection between the first, second, and new geometry (at which point, one of the tips may be selected or another tip geometry may be generated for comparison purposes).

In some embodiments, generating new geometries may include modifying the previously tested geometries for additional modeling. In some embodiments, the user may further modify a selected tip geometry to tune the geometry according to additional criteria. For example, the user may modify the length of the conical portion 2110 (or other geometric characteristics such as length of the shaft) to increase the twisting vibrations of the balanced tip 2100 to provide a greater lateral displacement of the distal end point 2106. In some embodiments, the user may try different locations and curvatures of one or more of the bends to reduce the lateral displacement toward the proximal end of the tip while increasing the lateral displacement toward the distal end of the tip. The modifications may be used for a third, fourth, etc., tip and the results compared to previous tip results to optimize the selection of the geometric characteristics of the tip.

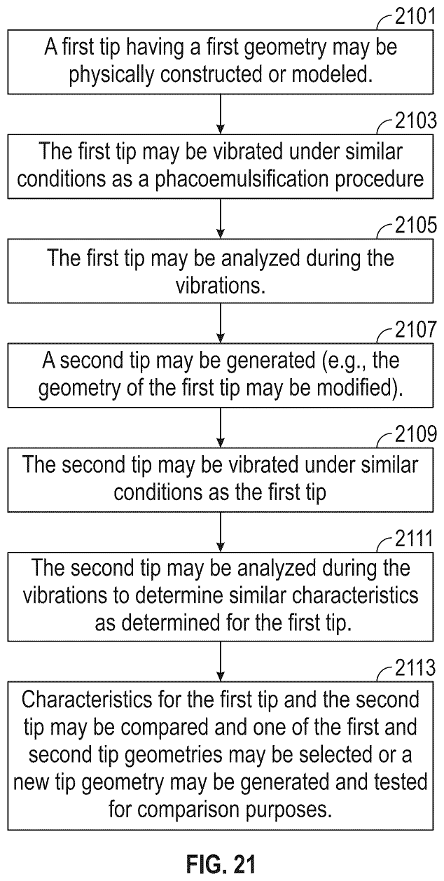

FIG. 21 illustrates a flowchart of another method for determining a tip geometry, according to an embodiment. The elements provided in the flowchart are illustrative only. Various provided elements may be omitted, additional elements may be added, and/or various elements may be performed in a different order than provided below.

At 2101, a first tip having a first geometry may be physically constructed or modeled (e.g., using Finite Element Analysis). In some embodiments, the first tip may have a circular cross section, square cross section, or a cross section that varies along an axis of the tip.

At 2103, the first tip may be vibrated under similar conditions as a phacoemulsification procedure (e.g., by being secured in a phacoemulsification handpiece as shown in FIGS. 10-11 and vibrated at a frequency of approximately 31 kHz and/or being “vibrated” using modeling software such as ANSYS). Other frequencies are also contemplated (e.g., approximately between 10 kHz and 60 kHz). In some embodiments, the first tip may be secured to a phacoemulsification handpiece to be vibrated. In some embodiments, the tip may be secured to a different apparatus (e.g., a test fixture) for applying the vibrations. In some embodiments, the end of the first tip may be placed in water or a material with similar characteristics as vitreous (other liquids are also contemplated). In some embodiments, the first tip may include two bends (e.g., 102, 103). Other numbers of bends are also contemplated.

At 2105, the first tip may be analyzed during the vibrations. For example, thermal imaging, stroboscopy, physical measurement of displacement, etc. may be used to determine lateral displacement (uₓ) and/or twist angle (ϕ) (or characteristics indicative of lateral displacement (uₓ) and/or twist angle (ϕ) for the tip. For example, in a thermal scan of the vibrating tip, locations of higher heat along the tip length may be indicative of larger lateral displacements (uₓ).

At 2107, a second tip may be constructed (e.g., the geometry of the first tip may be modified). Modifications may be made to different geometric characteristics as provided above. For example, the location and/or curvature of the second bend 2103 may be modified.

At 2109, the second tip may be vibrated under similar conditions as the first tip.

At 2111, the second tip may be analyzed during the vibrations to determine similar characteristics (such as lateral displacement and twist angle) as determined for the first tip.

At 2113, characteristics for the first tip and the second tip may be compared and one of the first and second tip geometries may be selected or a new tip geometry may be generated and tested for comparison purposes. For example, selecting the first tip geometry or the second tip geometry may be based on which tip geometry has a smaller lateral displacement along a portion of the tip shaft configured to be along an incision in an eye during a phacoemulsification procedure. In some embodiments, if the lateral displacement along a portion of the tip shaft configured to be along an incision in an eye during a phacoemulsification procedure (e.g., throughout a portion of the shaft that extends from the proximal end of the shaft (such as the end of the conical portion) to the first bend of the cutting edge portion) is greater than approximately 5% to 25% (e.g., 15%) (as noted above, other thresholds are also possible) of the displacement of the distal end point 2106 of the tip, a third tip may be generated and tested. The lateral displacement and/or twist angle of the third tip geometry may be determined and compared to the lateral displacement and/or twist angle of the first or second tip geometry for further selection between the first, second, and third tip geometries (at which point, one of the tips may be selected or another tip geometry may be generated for comparison purposes).

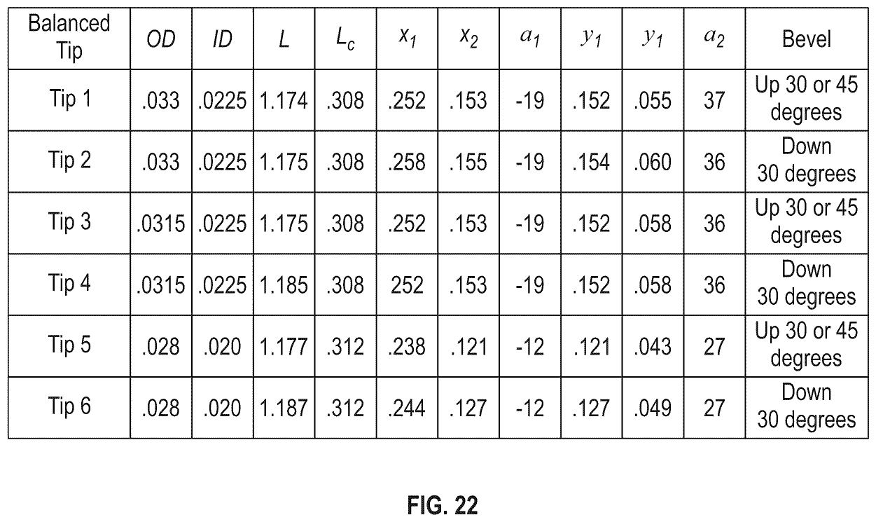

FIG. 22 illustrates six possible balanced tip embodiments (other embodiments are also possible). The balanced tip 2100 may have a geometry according to one of the sets of parameters provided in the FIG. 22 table. The balanced tip 2100 may have an outside diameter of OD inches; a diameter of the inside bore of ID inches; a total length of L inches from the hub (the proximal point of balanced tip 2100 that is configured to attach to the ultrasonic horn) to the cutting edge portion 2112 of balanced tip 2100. The conical portion 2110 of balanced tip 2100 may extend L_{c} inches from the hub. The first bend 2102 of balanced tip 2100 may have an angle of α₁ degrees and extend between points lying at a distance of x1 and x2 inches from the distal end point 2106. The second bend 2104 may have the angle of α₂ degrees and extend between points lying at a distance of x₁ and x₂ inches from the distal end point 2106. The cutting edge portion 2112 may have a beveled edge (i.e., at the distal most edge of the cutting edge portion) that is facing up 30 or 45 degrees or facing down 30 degrees as indicated in the table (as an example, the bevel shown in FIG. 16 is facing down). In some embodiments, a modeling system may include one or more processors. The processor may include single processing devices or a plurality of processing devices. Such a processing device may be a microprocessor, controller (which may be a micro-controller), digital signal processor, microcomputer, central processing unit, field programmable gate array, programmable logic device, state machine, logic circuitry, control circuitry, analog circuitry, digital circuitry, and/or any device that manipulates signals (analog and/or digital) based on operational instructions. The memory coupled to and/or embedded in the processors may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processors implement one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory may store, and the processor may execute, operational instructions corresponding to at least some of the elements illustrated and described in association with the figures (e.g., FIGS. 20 and 21).

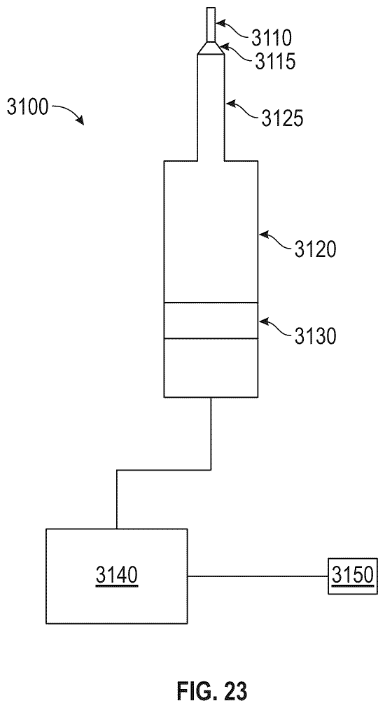

Referring now to FIG. 23, in some embodiments, the balanced tip discussed above may interchangeably be removed from the surgical handle system 1010 with, a system 3100, which is discussed in further detail below. In FIG. 23, hand piece system 3100 is coupled to console 3140. Console 3140 is coupled to foot switch 3150. Hand piece system 3100 has a cutting needle 3110, a horn 3120, and a set of piezoelectric crystals 3130. A needle interface 3115 connects cutting needle 3110 to a reduced diameter portion 3125 of horn 3 120. Needle 3110 is typically a thin needle made of titanium or stainless steel that is designed to emulsify a lens when vibrated ultrasonically. Needle 3110 is typically cylindrical in shape, has a small diameter of about 20-30 gauge, and has a length suitable for removal of a lens when inserted into the anterior chamber of the eye.

Horn 3120 is typically made of a rigid material suitable for medical use (such as a titanium alloy). Horn 3120 has a reduced diameter portion 3125 that is connected to a needle interface 3115. Needle interface 3115 typically has a threaded connection that accepts needle 3110. In this manner needle 3110 is screwed onto horn 3120 at needle interface 3115. Doing so provides a rigid connection between needle 3110 and horn 3120 so that vibration can be transmitted from horn 3120 to needle 3110.

Piezoelectric crystals 3130 supply ultrasonic vibrations that drive both the horn 3120 and the attached cutting needle 3110 during phacoemulsification. Piezoelectric crystals 3130 are affixed to horn 3120. Crystals 3130 are typically ring shaped, resembling a hollow cylinder and constructed from a plurality of crystal segments. When excited by a signal from console 3140, crystals 3130 resonate, producing vibration in horn 3120.

Console 3140 includes a signal generator that produces a signal to drive piezoelectric crystals 3130. Console 3140 has a suitable microprocessor, micro-controller, computer, or digital logic controller to control the signal generator. In operation, console 3140 produces a signal that drives piezoelectric crystals 3130. Piezoelectric crystals 3130, when excited, cause horn 3120 to vibrate. Needle 3110, connected to horn 3120, also vibrates. When needle 3110 is inserted into the anterior chamber of the eye and vibrated, it acts to emulsify a cataractous lens.

In cataract surgery, the needle 3110 is typically made only of titanium or stainless steel. Because the needle 3110 is vibrated in the eye ultrasonically (typically at frequencies greater than 30 kHz), it is important to have a needle 3110 that can withstand such vibrations. It had been thought that polymer needles or needles with a polymer over mold would not withstand such vibrating force. The inventors of the present application have discovered that the phacoemulsification needles described and claimed herein withstand such vibrations and function to remove natural lenses during cataract surgery.

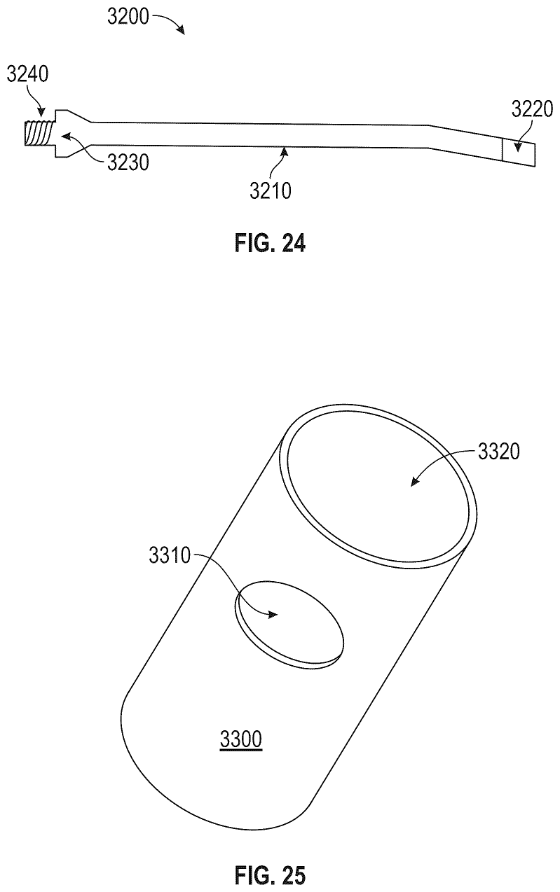

FIG. 24 is a side view of a phacoemulsification needle 3200 with a polymer distal end (i.e., tip) according to an embodiment of the present invention. Needle 3200 has a shaft 3210 terminating at a distal end 3220. Opposite the polymer distal end 3220 is a hub 3230 and threaded connection 3240. Shaft 3210 is typically cylindrical with a central bore that forms a part of the aspiration path. Fragmented lens particles and irrigating solution are aspirated through the central bore of shaft 3210. Hub 3230 and threaded connection 3240 allow the needle 3200 to be coupled to a hand piece. Shaft 3210, hub 3230, and threaded connection 3240 are typically made from titanium, stainless steel or other similar material. As described in greater detail below, distal end 3220 may be made of a polymer, plastic, silicone or other similar material. Such materials are generally softer, smoother, and have more rounded edges than the titanium or stainless steel of traditional phacoemulsification needles. As noted, the distal end 3220 of phacoemulsification needle 3200 is inserted into the eye to remove a cataract. The distal end 3220 of phacoemulsification needle 3200 also comes into contact with other delicate eye structures. A distal end made of a polymer, plastic, silicone or other similar material tends to damage these delicate eye structures less than a phacoemulsification needle with a distal end made of titanium or stainless steel.

FIG. 25 is a perspective view of the distal end of a phacoemulsification needle according to an embodiment of the present invention. FIG. 25 depicts the distal end 3300 of a phacoemulsification needle with a central bore 3320 and a through hole 3310 in a side wall of distal end 3300. Distal end 3300 is typically made of titanium, stainless steel, or other similar material. The through hole 3310 allows an over mold to be secured to distal end 3300. In some cases, a polymer may be over molded onto distal end 3300. Through hole 3310 is filled with the polymer and acts to secure it to distal end 3300. While through hole 3310 is shown as a continuous oval opening in a side wall of distal end 3300, through hole may be of any suitable shape. In addition, through hole 3310 may have a discontinuous periphery (e.g., serrated) that serves to grip a polymer over mold.

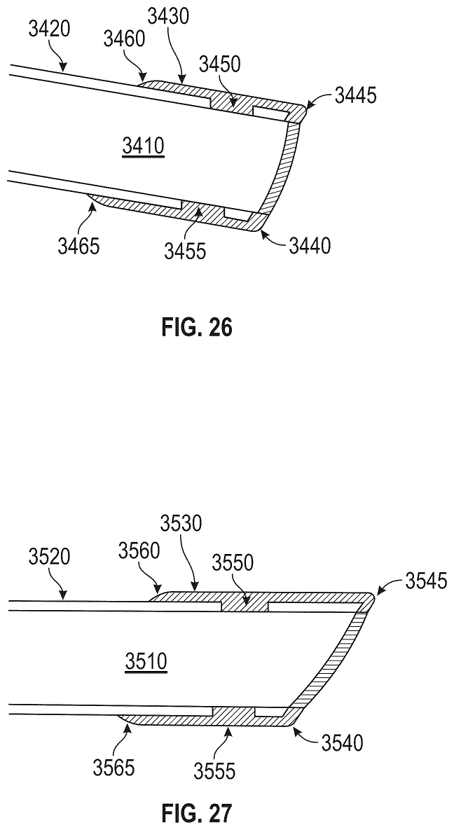

FIG. 26 is a side cross section view of the distal end of a phacoemulsification needle with an over molded distal end (i.e., tip) according to an embodiment of the present invention. Over mold 3430 (shown as the shaded portion of FIG. 26) is located on shaft 3420. Shaft 3420 has a continuous central bore that extends to an open end at the distal end of over mold 3430. The distal end of over mold 3430 has a rounded front edge (3440, 3445) that extends circumferentially around the perimeter of shaft 3420. When shaft 3420 is a hollow cylinder, over mold 3430 is located around the periphery of the cylinder and covers the edges of the distal end of the cylinder. Typically, a phacoemulsification needle is essentially a metal tube with a wall thickness.

The central bore of the metal tube extends completely and continuously through it. The distal end of such a phacoemulsification needle often has sharp edges (or edges that are not rounded). The depth of these edges is defined by the wall thickness of the needle. Over mold 3430 would be located on such a phacoemulsification needle around its periphery. Over mold 3430 would extend to cover the edges on the distal end of the needle. In this manner, over mold 3430 provides a soft, rounded, pliable, and/or smooth surface that covers the sharp edges of the needle and extends back from the distal end of the needle along the needle shaft 3420.

Over mold 3430 has a rounded or smooth front edge (shown in the cross section drawing as 3440 and 3445) and a rounded or smooth trailing edge (shown in the cross section drawing as 3460 and 3465). These front (3440, 3445) and trailing (3460, 3465) edges allow for the phacoemulsification needle to be easily inserted into and removed from a small incision in the eye. Because these front edges (3440, 3445) and trailing edges (3460, 3465) are smooth, soft, rounded and/or pliable, over mold 3430 better protects delicate eye structures during cataract surgery. In particular, the front edge (3440, 3445) or over mold 3430 is much less likely to damage eye structures than a traditional phacoemulsification needle.

Over mold 3430 may be made of a polymer, plastic, silicone or the like. Generally, over mold 3430 is molded onto shaft 3420 by, for example, an injection molding process. As shown in FIG. 26, two through holes 3450 and 3455 secure over mold 3430 to shaft 3420. In other examples of the invention, other structures on shaft 3420 (such as protrusions, indentations, or the like) are used to secure over mold 3430 to shaft 3420. In other examples, no through holes 3450, 3455 are present, and over mold 3430 is secured onto shaft 3420 by friction or an adhesive. In one example, over mold 3430 is premolded and fixed to shaft 3420 by friction or an adhesive. When over mold 3430 is injection molded onto shaft 3420, a dowel or other similar structure may be placed in central bore 3410 to prevent material from entering the central bore 3410 through the through holes 3450, 3455.

Referring now to FIG. 27, FIG. 27 is a side cross section view another example of the distal end of a phacoemulsification needle with an over molded distal end (i.e., tip) according to an embodiment of the present invention. In FIG. 76, the needled is beveled. Over mold 3530 (shown as the shaded portion of FIG. 5) is located on shaft 3520. Shaft 3520 has a continuous central bore that extends to an open end at the distal end of over mold 3530. The distal end of over mold 3530 has a rounded front edge (3540, 3545) that extends circumferentially around the perimeter of shaft 3520. When shaft 3520 is a hollow cylinder, over mold 3530 is located around the periphery of the cylinder and covers the edges of the distal end of the cylinder. Typically, a phacoemulsification needle is essentially a metal tube with a wall thickness. The central bore of the metal tube extends completely and continuously through it. The distal end of such a phacoemulsification needle often has sharp edges (or edges that are not rounded) especially when the end of the needle is beveled. The depth of these edges is defined by the wall thickness of the needle. Over mold 3530 would be located on such a phacoemulsification needle around its periphery. Over mold 3530 would extend to cover the edges on the distal end of the needle. In this manner, over mold 3530 provides a soft, rounded, pliable, and/or smooth surface that covers the sharp edges of the needle and extends back from the distal end of the needle along the needle shaft 3520.

Over mold 3530 has a rounded or smooth front edge (shown in the cross section drawing as 3540 and 3545) and a rounded or smooth trailing edge (shown in the cross section drawing as 3560 and 3565). These front edges (3540, 3545) and trailing edges (3560, 3565) allow for the phacoemulsification needle to be easily inserted into and removed from a small incision in the eye. Because these front (3540, 3545) and trailing (3560, 3565) edges are smooth, soft, rounded and/or pliable, over mold 3530 better protects delicate eye structures during cataract surgery. In particular, the front edge (3540, 3545) or over mold 3530 is much less likely to damage eye structures than a traditional phacoemulsification needle.

Over mold 3530 may be made of a polymer, plastic, silicone or the like. Generally, over mold 3530 is molded onto shaft 3520 by, for example, an injection molding process. As shown in FIG. 27, two through holes 3550 and 3555 secure over mold 3530 to shaft 3520. In other examples of the invention, other structures on shaft 3520 (such as protrusions, indentations, or the like) are used to secure over mold 3530 to shaft 3520. In other examples, no through holes 3550, 3555 are present, and over mold 3530 is secured onto shaft 3520 by friction or an adhesive. In one example, over mold 3530 is premolded and fixed to shaft 3520 by friction or an adhesive. When over mold 3530 is injection molded onto shaft 3520, a dowel or other similar structure may be placed in central bore 3510 to prevent material from entering the central bore 3510 through the through holes 3550, 3555.

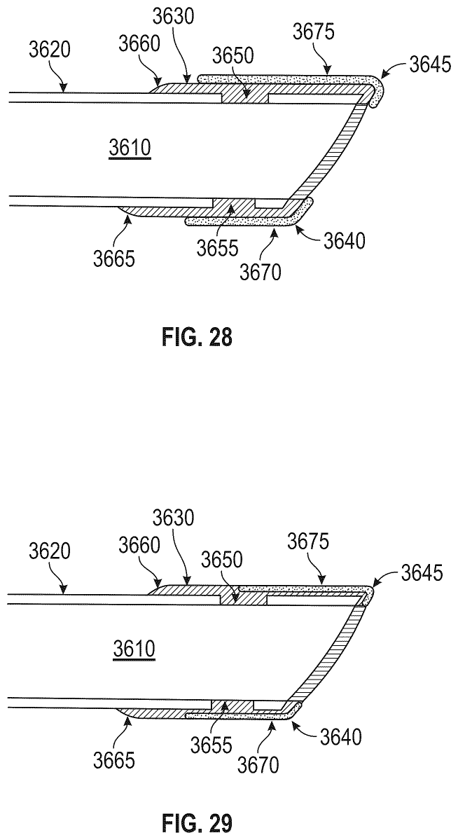

FIGS. 28 and 29 are side cross section views of the distal end of a phacoemulsification needle with an over molded distal end (i.e., tip) according to an embodiment of the present invention. In FIGS. 28 and 29, a first over mold 3630 and a second over mold (3570, 3575) are present on the distal end of shaft 3620. First over mold 3630 (shown as the shaded portion of FIG. 6) is located on shaft 3620. Shaft 3620 has a continuous central bore that extends to an open end at the distal end of first over mold 3630. The distal end of first over mold 3630 has a rounded front edge (3640, 3645) that extends circumferentially around the perimeter of shaft 3620. When shaft 3620 is a hollow cylinder, first over mold 3630 is located around the periphery of the cylinder and covers the edges of the distal end of the cylinder. Typically, a phacoemulsification needle is essentially a metal tube with a wall thickness. The central bore of the metal tube extends completely and continuously through it. The distal end of such a phacoemulsification needle often has sharp edges (or edges that are not rounded) especially when the end of the needle is beveled. The depth of these edges is defined by the wall thickness of the needle. First over mold 3630 would be located on such a phacoemulsification needle around its periphery.

First over mold 3630 would extend to cover the edges on the distal end of the needle. In this manner, first over mold 3630 provides a soft, rounded, pliable, and/or smooth surface that covers the sharp edges of the needle and extends back from the distal end of the needle along the needle shaft 3620. A second over mold 3670, 3675 is located on first over mold 3630. Second over mold 3670, 3675 may cover all or a portion of first over mold 3630. In one example, second over mold 3670, 3675 is silicone which provides a smooth and pliable surface that does not cause unwanted damage to eye structures. In this case, the second over mold 3670, 3675 may be applied to first over mold 3630 in a two shot molding process, by an adhesive, or by other similar means. In the embodiments shown in FIGS. 28 and 29, second over mold 3670, 3675 extends along shaft 3620 and covers the distal edge of first over mold 3630. In such a case, the sharp edges of the distal end of shaft 3620 are covered by the material of first over mold 3630 and the material of second over mold 3670, 3675. In the example of FIG. 28 the second over mold 3670, 3675 is applied on top of first over mold 3630. In the example of FIG. 29 second over mold 3670, 3675 is embedded in first over mold 3630 such that the outer surface of first over mold 3630 and second over mold 3670, 3675 form a smooth, continuous surface.

First over mold 3630 and second over mold 3670, 3675 have rounded or smooth front edges 3640, 3645. First over mold has a rounded or smooth trailing edge (shown in the cross section drawing as 3660 and 3665). These front (3640, 3645) and trailing (3660, 3665) edges allow for the phacoemulsification needle to be easily inserted into and removed from a small incision in the eye. Because these front (3640, 3645) and trailing (3660, 3665) edges are smooth, soft, rounded and/or pliable, first over mold 3630 and second over mold 3670, 3675 better protect delicate eye structures during cataract surgery. In particular, the front edge (3640, 3645) or first over mold 3630 and second over mold 3670, 3675 are much less likely to damage eye structures than a traditional phacoemulsification needle.

First over mold 3630 may be made of a polymer, plastic, silicone or the like. Generally, first over mold 3630 is molded onto shaft 3620 by, for example, an injection molding process. As shown in FIG. 28, two through holes 3650 and 3655 secure first over mold 3630 to shaft 3620. In other examples of the invention, other structures on shaft 3620 (such as protrusions, indentations, or the like) are used to secure first over mold 3630 to shaft 3620. In other examples, no through holes 3650, 3655 are present, and first over mold 3630 is secured onto shaft 3620 by friction or an adhesive. In one example, first over mold 3630 is premolded and fixed to shaft 3620 by friction or an adhesive. When first over mold 3630 is injection molded onto shaft 3620, a dowel or other similar structure may be placed in central bore 610 to prevent material from entering the central bore 610 through the through holes 3650, 3655.

In operation, any of the needles 3110, 3200 can be secured to a phacoemulsification hand piece via a threaded connection 3240. The needle 3110, 3200 is then inserted into the anterior chamber of the eye through a small incision and vibrated ultrasonically. Lens material and fluid are aspirated through the central bore 3410, 3510, or 3610 of the respective needle 3110, 3200. Over mold 3430, 3530, or 3630 is secured to the needle such that the ultrasonic vibrations do not cause it to move. In other words, the over mold is subjected to the stresses of vibration and surgery without being dislocated from the needle. In addition, the front edge of the over mold protects delicate eye structures from the unintended stresses of surgery. For example, the over mold is rigid enough to fracture a natural lens but smooth enough not to damage the posterior lens capsule. When a second over mold is present, the first and second over molds operate cooperatively.

In some embodiments, controller 1060 may utilize use Fast Fourier Transform (FFT) for tuning the operation of the surgical handpiece system 1010, as described in further detail below. Fast Fourier Transform (FFT) is a powerful mathematical technique that enables the analysis of complex signals and waveforms by converting them from the time domain to the frequency domain. In the context of our surgical handpiece for ocular surgery, FFT plays a pivotal role in actively tuning the applied frequencies of the torsional and longitudinal vibrations of the phacoemulsification tip. By utilizing FFT, the embodiments herein may advantageously implement the resonant frequencies of the tip's motion, enabling efficient and controlled performance during surgical procedures.

For example, to implement FFT for tuning, controller 1060 of the surgical handpiece system 1010 collects real-time voltage and current data from the ultrasonic drive system, which generates the vibrations in the phacoemulsification tip. This data represents the time-domain signals of the vibrations. The collected data is then subjected to the FFT algorithm, which decomposes the complex time-domain signals into their constituent frequencies. Through this analysis, all resonances can be extracted, including the resonant frequencies of both torsional and longitudinal vibrations as well as alternate modes of oscillation besides torsional and longitudinal modes, providing valuable insights into the behavior of the tip during surgery.

With the resonant frequencies identified through FFT, controller 1060 actively adjusts the frequencies applied to the phacoemulsification tip. The system is designed to ensure that the applied frequencies align with the identified resonant frequencies, maximizing the efficiency of the tip's motion. This active frequency adjustment ensures that the vibrations are precisely tuned to avoid the risk of harmful over-or under-vibration and enables the tip to perform optimally, promoting safety and efficacy during ocular surgery.

The implementation of FFT for tuning not only provides precise frequency control of any modes of oscillation, simultaneously, but also enables real-time adaptation to changing surgical conditions. Controller 1060 continuously monitors the vibrations and adjusts the applied frequencies accordingly. If the surgical environment experiences fluctuations in factors such as fluid levels, temperature, or occlusions, the system can quickly respond and recalibrate the vibrations to maintain consistent performance. This adaptive capability enhances the handpiece's versatility and ensures seamless integration into a wide range of ocular surgical scenarios.

The surgeon benefits from the implementation of FFT for tuning as well. The user interface of the handpiece allows the surgeon to monitor the vibrations and receive feedback on the resonant frequencies in real-time. Surgeons can make precise adjustments to the applied frequencies through the user-friendly interface, tailoring the tip's motion according to their preferences or specific surgical requirements. This control enhances the precision and dexterity of the surgical procedure, potentially leading to improved surgical outcomes and a more efficient overall process.

To begin the process, controller 1060 captures the time-domain voltage and current signals in real-time. The time-domain signals represent the dynamic behavior of the vibrations over time. Applying FFT to these signals involves decomposing them into their individual frequency components. This transformation allows the system to identify the frequencies that contribute most significantly to the vibrations, revealing the resonant frequencies for both torsional and longitudinal motions as well as other modalities of motion.

After extracting the resonant frequencies from the FFT analysis, controller 1060 calculates the impedance associated with each mode of vibration. Impedance is a crucial parameter that characterizes how a system responds to an applied force or voltage. In the context of the surgical handpiece, impedance provides information about the resistance of the phacoemulsification tip to torsional and longitudinal vibrations. By quantifying the impedance for each mode, the system gains a deeper understanding of the tip's mechanical behavior and its responsiveness to different frequencies.

With the impedance values determined, controller 1060 takes an active role in tuning the applied frequencies of the torsional and longitudinal vibrations. By modulating the frequency and amplitude of the applied signals, a system controller (e.g., controller 1060) can ensure that the vibrations closely match the identified resonant frequencies. Such precise tuning is advantageous for achieving maximum vibrational amplitudes and cutting efficiency while minimizing unnecessary energy expenditure.

By actively adjusting the applied frequencies based on the resonant frequencies and impedance characteristics, the embodiments described herein achieve dynamic and adaptive performance. The system continually optimizes the applied vibrations in real-time, accounting for variations in the surgical environment and other factors that may influence the tip's behavior. This level of tuning precision ensures that the handpiece maintains its performance efficiency throughout the surgical procedure, providing surgeons with enhanced control and promoting superior patient outcomes. Additionally, the embodiments described herein provide the ability to extract multiple resonances, or modalities of motion, simultaneously. This includes harmonics of motion, as further described below.

In some embodiments, use of harmonics may be implemented for advantageously driving the surgical handpiece. The implementation of harmonics to drive the phacoemulsification tip in the surgical handpiece presents an advantageous approach to enhance the efficiency and versatility of the device. Harmonics are multiples of the fundamental frequencies and can significantly impact the motion characteristics of the tip. By generating additional frequencies that include harmonics of the main resonant frequencies for both torsional and longitudinal vibrations, the embodiments describe herein may create complex waveforms that optimize the emulsification process during ocular surgery.

For example, to use harmonics for driving the tip, controller 1060 of the surgical handpiece system 1010 may be configured to generate additional frequencies that correspond to harmonics of the fundamental resonant frequencies. Such additional frequencies are superimposed on the main torsional and longitudinal vibrations. The combination of the fundamental frequencies and their harmonics creates rich and dynamic motion patterns for the phacoemulsification tip, maximizing its cutting efficiency and overall performance.

By incorporating harmonics into the motion of surgical handpiece working tip, the handpiece system becomes capable of delivering precise and controlled energy to the surgical site. The harmonics complement the fundamental vibrations, allowing for finer and more efficient emulsification of cataractous lens material. This enhancement in emulsification efficiency may reduce the overall energy required during the procedure, mitigating the risk of potential damage to surrounding tissues and promoting faster patient recovery.

Thus the embodiments herein offer a flexible and customizable approach to utilizing harmonics. Surgeons have the option to adjust the amplitude and phase relationships of the harmonics relative to the fundamental frequencies. This feature enables the creation of various harmonic profiles tailored to specific surgical needs. Surgeons can experiment with different harmonic combinations to optimize the tip 1020, 21 motion for various lens densities, sizes, and individual patient characteristics.

While harmonics offer the potential for enhanced performance, controller 1060 of the handpiece is designed to ensure safety and precision. Safety limits and feedback mechanisms are implemented to prevent excessive harmonic amplitudes that could lead to undesirable vibrations. Surgeons can rely on the precision control of the handpiece to fine-tune harmonic settings, promoting an optimal balance between efficiency and safety throughout the entire ocular surgical procedure. The incorporation of harmonics into the tip motion is an innovative feature that significantly elevates the capabilities of the surgical handpiece, contributing to improved surgical outcomes and patient care.

In some embodiments, additional modes of tip motion may be implemented in addition to longitudinal and torsionally as discussed above. For example, additional modes of tip motion beyond the torsional and longitudinal vibrations. These additional modes open up new possibilities for enhancing the precision and effectiveness of ocular surgery. By incorporating the applicable resonant frequencies for these modes, one or more embodiments may capitalize on the natural vibrational behavior of the phacoemulsification tip, resulting in improved emulsification performance and surgical outcomes.

For example, in some embodiments, the system 1010, 3100 includes a diverse set of additional modes of tip motion, carefully selected based on their relevance to ocular surgery. These modes are engineered to cater to different stages of the emulsification process and specific surgical needs. Examples of additional modes may include transverse oscillations, radial vibrations, and any other modes identified as beneficial for particular surgical situations.

Incorporating the applicable resonant frequencies for each additional mode is advantageous to optimizing the performance of handpiece system 1010, 3100. Rigorous testing and analysis have been conducted to identify the natural frequencies at which the tip exhibits maximum amplitude and efficiency for each mode. These resonant frequencies are precisely targeted and utilized in conjunction with controller 1060 to drive the tip motion with exceptional accuracy and efficacy.

It has been shown that the combination of the conventional torsional and longitudinal vibrations with the additional modes, each utilizing its applicable resonant frequencies, creates synergistic motion profiles. Such profiles can be tailored to suit various surgical scenarios, such as emulsification of different lens types or addressing varying degrees of cataract density. Surgeons have the flexibility to select and activate specific motion profiles to meet the unique requirements of each surgical case, promoting a highly personalized and efficient approach to ocular surgery.

The integration of additional modes of tip motion, together with the use of the applicable resonant frequencies, brings substantial advantages to the surgical handpiece system 1010, 3100. Surgeons can now achieve improved lens fragmentation, reducing the overall phacoemulsification time and minimizing potential trauma to ocular tissues. Moreover, the ability to leverage the resonant frequencies of each mode ensures an optimal balance between energy efficiency and cutting efficacy, ultimately contributing to enhanced patient safety and quicker post-operative recovery.

Some embodiments may include the incorporation of more than two resonant frequencies in surgical handpiece system 1010, 3100 which advantageously introduces a new level of vibrational diversity and adaptability during ocular surgery. By extending beyond the conventional torsional and longitudinal vibrations, the handpiece can now harness the power of multiple resonant frequencies. Each frequency corresponds to distinct modes of motion, enabling surgeons to leverage a broader range of cutting patterns and dynamics. This enhanced vibrational diversity allows for a more precise and controlled emulsification process, accommodating various lens densities and anatomical conditions encountered in complex cataract surgeries.

With the capability to apply three or more different modes of tip motion, each driven by their specific resonant frequencies, the surgical handpiece can generate multifaceted surgical profiles. Surgeons can tailor the hand piece's motion to suit the specific requirements of each surgical case. For instance, denser cataracts may benefit from motion profiles that emphasize longitudinal vibrations, while softer or fragmented lenses may necessitate a stronger emphasis on torsional vibrations. The ability to dynamically switch between resonant frequencies allows for seamless transitions between motion profiles, offering an unprecedented level of versatility in ocular surgery.

An advantage of utilizing more than two resonant frequencies includes the potential for dynamic frequency modulation. Controller 1060 of the handpiece may continuously adjust the applied frequencies based on real-time feedback and surgical conditions. Surgeons have the flexibility to fine-tune the intensity and distribution of each resonant frequency during the procedure, optimizing the handpiece's performance to address unexpected challenges or changes in the surgical environment. This dynamic frequency modulation ensures that the handpiece remains adaptable, responsive, and precise, elevating the surgeon's control and enhancing patient safety.

The incorporation of multiple resonant frequencies also has implications for energy efficiency during surgery. By strategically utilizing resonant frequencies that align with the natural oscillatory behavior of the tip, the handpiece can achieve maximum amplitude and cutting efficiency with minimal energy consumption. This optimized energy utilization not only reduces the overall power requirements of the handpiece but also contributes to a reduction in thermal energy production. As a result, there is a potential reduction in the risk of thermal injury to the surrounding ocular tissues, further enhancing the safety profile of the surgical handpiece. Thus, the integration of more than two resonant frequencies represents a significant advancement in ocular surgery. Such innovative features as described above empowers surgeons with greater control, precision, and flexibility, leading to improved surgical outcomes and enhanced patient care.

In some embodiments, the surgical handpiece system (e.g., 1010, 3100) is capable to variably modulate the percentage of each mode and resonant frequency applied, which offers surgeons an unprecedented level of adaptive motion control during ocular surgery. Such features allow for real-time adjustments to the distribution of vibrational modes, enabling surgeons to fine-tune the balance between torsional and longitudinal vibrations based on the specific surgical requirements and patient characteristics. Surgeons can dynamically alter the percentage of each mode to optimize cutting efficiency, enhance emulsification performance, and address varying degrees of lens density, providing a highly personalized and responsive approach to each surgical case.

For example, having the ability to fine-tune the percentages of torsional and longitudinal vibrations applied to the phacoemulsification tip, a user interface provides an intuitive platform for the surgeon to interactively adjust these percentages in real-time. For instance, the surgeon can opt to increase the proportion of torsional motion for more efficient lens fragmentation or enhance the contribution of longitudinal motion for improved aspiration of emulsified lens material. The flexibility to customize the motion profile empowers the surgeon to tailor the handpiece's behavior to align with their unique surgical technique and expertise, fostering a personalized and optimized approach to ocular surgery

Additionally, the variable and modulating feature plays a crucial role in adapting to changes in surgical conditions. During the course of the surgery, various factors such as an occlusion, changes in aspiration rate, irrigation rate, or temperature may arise, altering the dynamics of the surgical environment. The system monitors these changing conditions in real-time and dynamically adjusts the ratio of motion to ensure handpiece system 1010, 3100 remains responsive and effective.

For example, if an occlusion occurs, system 1010, 3100 may automatically increase the proportion of longitudinal motion to facilitate efficient aspiration and prevent tissue damage. Similarly, changes in aspiration or irrigation rates may warrant adjustments to the vibrational distribution to optimize cutting performance and manage fluid dynamics effectively. This adaptability to changing surgical conditions contributes to a smoother and safer surgical experience, reducing the likelihood of complications and enhancing patient care.

By combining user-controlled customization and adaptive responsiveness to surgical conditions, the variable and modulating feature showcases the handpiece's versatility and precision. Surgeons benefit from unparalleled control over the vibrational behavior, enabling them to navigate through different surgical challenges with case and confidence. Ultimately, this feature elevates the overall performance of the surgical handpiece, promoting enhanced surgical outcomes and reinforcing the handpiece's position as a state-of-the-art tool in modern ocular surgery.

In some embodiments, the user interface of the handpiece empowers surgeons to interactively adjust the percentage of each mode or resonant frequency according to their preferences or surgical conditions. Surgeons can easily navigate through the user-friendly controls to fine-tune the motion profile and achieve an ideal combination of vibrational modes tailored to their unique surgical technique and comfort. This user-centric customization not only enhances surgical control but also fosters surgeon confidence and satisfaction, translating into improved patient outcomes.

The variable modulation of each mode and resonant frequency aligns with optimizing both efficiency and safety in ocular surgery. Surgeons may regulate the vibrational distribution to minimize unnecessary energy expenditure while maximizing the effectiveness of the cutting process. By tailoring the motion profile to suit the specific surgical context, there is a potential reduction in the risk of tissue damage, thermal injury, and post-operative complications. This adaptability fosters a safer surgical environment, enhancing patient well-being and recovery.

As surgery progresses and the surgical environment evolves, the ability to modulate vibrational modes becomes especially valuable. System 1010, 3100 constantly monitors the surgical conditions, such as fluid dynamics and tissue density, and dynamically adjusts the percentage of each mode accordingly. Surgeons can rely on this adaptive feature to respond to unexpected challenges during the procedure, ensuring that the handpiece remains optimized for performance and adaptively aligned with the surgical context.

The variable modulation of each mode and resonant frequency represents a groundbreaking advancement in surgical technology, setting a new standard for precision and control in ocular surgery. By combining this feature with the handpieces other innovative functionalities, such as additional modes of tip motion and harmonics, the embodiments herein set a new benchmark in the landscape of ocular phacoemulsification and offer improve surgical outcomes.

From the above, it may be appreciated that the present invention provides an improved phacoemulsification tip or needle for cataract surgery. The present invention provides a phacoemulsification tip or needle with a polymer distal end. The present invention is illustrated herein by example, and various modifications may be made by a person of ordinary skill in the art.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

### Example Embodiments

Embodiment 1: A method of operating an ultrasonic handpiece comprising a piezoelectric element assembly, the method comprising: applying control signals to drive the piezoelectric element assembly simultaneously in a first mode of oscillation and a second mode of oscillation; generating feedback of a resulting oscillation of the piezoelectric element assembly in the first mode and the second mode; and adjusting, based on the feedback, the control signals so that the resulting oscillation in the first mode and the resulting oscillation in the second mode are each approximately at a respective resonant frequency.

Embodiment 2: The method of Embodiment 1, wherein the generating feedback comprises determining a root mean square (RMS) value of measured voltage and resulting current in each of the first mode and the second mode.

Embodiment 3: The method of Embodiment 1, wherein the generating feedback comprises determining a magnitude of a composite voltage and a composite current of the resulting oscillation.

Embodiment 4: The method of Embodiment 1, wherein the generating feedback comprises determining a phase of a composite voltage and a composite current of the resulting oscillation.

Embodiment 5: The method of Embodiment 1, wherein the piezoelectric element assembly comprises one or more pairs of ring-shaped piezoelectric elements.

Embodiment 6: The method of Embodiment 1, wherein the ultrasonic handpiece comprises an additional piezoelectric element assembly, and wherein each piezoelectric element assembly is spaced apart along a longitudinal axis of the ultrasonic handpiece.

What is claimed is:

1. A surgical handpiece for ocular surgery, comprising:
a phacoemulsification tip attached to a shaft;
a fluid management system configured to manage inflow and outflow of fluid from an ocular surgical site;
an ultrasonic drive system configured to generate torsional and longitudinal vibrations in the phacoemulsification tip; and
a controller configured to:
analyze applied voltage and current to the ultrasonic drive system using Fast Fourier Transforms (FFTs), and
tune applied frequencies of the torsional and longitudinal vibrations according to at least one resonant frequency.

2. The surgical handpiece of claim 1, wherein the phacoemulsification tip comprises a cutting edge portion having at least a first bend and a second bend.

3. The surgical handpiece of claim 2, wherein a geometry of the shaft, the first bend, and the second bend is configured to produce a lateral displacement, perpendicular to the shaft, during ultrasonic torsional vibration of the phacoemulsification tip, of less than approximately 5% to 25% of the lateral displacement at a distal end point of the phacoemulsification tip throughout a portion of the shaft that extends from a proximal end of the shaft to the first bend of the cutting edge portion.

4. The surgical handpiece of claim 1, wherein the phacoemulsification tip comprises a first over mold located on an exterior surface and a distal edge of the shaft, the first over mold covering at least a portion of a periphery of the exterior surface of the shaft, the first over mold covering the distal edge and terminating at a central bore of the shaft.

5. The surgical handpiece of claim 1, further comprising a user interface for adjusting a ratio of torsional to longitudinal vibrations applied to the phacoemulsification tip.

6. The surgical handpiece of claim 1, wherein the controller is further configured to apply additional frequencies, including harmonics of resonant frequencies, for driving the phacoemulsification tip.

7. The surgical handpiece of claim 1, wherein the controller is further configured to modulate a percentage of each mode or resonant frequency applied based on surgical conditions.

8. The surgical handpiece of claim 1, wherein the fluid management system is configured to adapt to changes in an aspiration rate, an irrigation rate, or a temperature at a surgical site.

9. The surgical handpiece of claim 1, wherein the surgical handpiece is configured to control the fluid management system during a surgical procedure.

10. The surgical handpiece of claim 1, wherein the ultrasonic drive system is configured to apply additional modes of tip motion beyond torsional and longitudinal vibrations.

11. The surgical handpiece of claim 1, wherein the controller is configured to actively tune the applied frequencies of the torsional and longitudinal vibrations according to respective resonant frequencies.

12. The surgical handpiece of claim 1, wherein the surgical handpiece is configured to modulate a percentage of each mode of vibration that is applied to the phacoemulsification tip, and adjust the percentage of each mode of vibration that is applied to the phacoemulsification tip according to a user preference or one or more surgical conditions.

13. A method of operating an ultrasonic handpiece, the method comprising:
applying control signals to drive a piezoelectric element assembly of the ultrasonic handpiece simultaneously in a first mode of oscillation and a second mode of oscillation;
generating feedback of a resulting oscillation of the piezoelectric element assembly in the first mode and the second mode; and
adjusting, based on the feedback, the control signals so that the resulting oscillation in the first mode and the resulting oscillation in the second mode are each approximately at a respective resonant frequency.

14. The method of claim 13, wherein the first mode of oscillation comprises a longitudinal motion and the second mode of oscillation comprises a torsional motion.

15. The method of claim 13. wherein the ultrasonic handpiece comprises a phacoemulsifier.
